(19) United States
(12) Patent Application Publication
(10) Pub. No.: US 2007/0196346 A1
(43) Pub. Date: Aug. 23, 2007

(54) Poxvirus Vector Encoding Prostate Specific Antigens For Treatment Of Prostate Cancer

(76) Inventors: Michael Paul Brown, St. Georges (AU); Paul Michael Howley, Glen Waveley (AU)

Correspondence Address: SCULLY, SCOTT, MURPHY & PRESSER, P.C., 400 GARDEN CITY PLAZA, SUITE 300, GARDEN CITY, NY, 11530, US

(21) Appl. No.: 10/569,157

(22) PCT Filed: Aug. 20, 2004
(86) PCT No.: PCT/AU04/01129

§ 371 Date: Oct. 13, 2006

(30) Foreign Application Priority Data
Aug. 21, 2003 (AU) 2003904496

(51) Int. Cl. A61K 48/00; A61K 38/20; A61K 38/19; C12Q 1/70; C12Q 1/68; C07H 21/04; A61K 38/21; C12N 15/86

(52) U.S. Cl. 424/93.21; 424/85.1; 424/85.2; 424/85.5; 435/456; 536/24.3; 435/5; 435/6; 435/325; 536/23.2

(57) ABSTRACT

A genetic vaccine construct comprising a poxvirus vector which incorporates and, on administration to a subject, expresses in a cell of said subject a sequence of nucleotides encoding a prostate-specific polypeptide, wherein said poxvirus vector does not productively infect said subject. The genetic vaccine construct is for treatment of prostate cancer. The prostate specific polypeptide is preferably rat prostatic acid phosphatase, and is preferably xenogenic to the subject. The poxvirus vector is an avipox virus vector, preferably a fowl poxvirus vector. The genetic vaccine construct may also encode for one or more cytokines.

### FIELD OF THE INVENTION

The present invention relates generally to the field of genetic vaccinations and particularly to genetic immunotherapy and/or immunoprophylaxis of prostate cancer. More particularly, the present invention provides a genetic construct capable of stimulating a selective immune response to prostate cells including prostate cancer cells. The present invention also provides, inter alia, compositions for the immunotherapy and/or immunoprophylaxis of prostate cancer, antibodies thereto and diagnostic reagents therefor and methods for the treatment and/or prophylaxis of prostate cancer.

### BACKGROUND OF THE INVENTION

Bibliographic details of the publications referred to by author in this specification are collected at the end of the description.

Reference to any prior art in the specification is not, and should not be taken as, an acknowledgment or any form of suggestion that this prior art forms part of the common general knowledge in any country.

Advances in molecular biology and informatics in the last decade have greatly enhanced our broad understanding, and potential to gain a greater understanding, of biological events and have created enormous potential for pharmaceutical and related industries to develop strategies for the prevention and treatment of diseases and other disorders. A particularly important problem relates to the prevention and treatment of prostate cancer and other prostate related diseases or conditions.

Prostate cancer is the second most common cause of cancer death in males. Prostate cancer is only potentially curable when it is confined to the prostate gland using one of two local modalities of treatment: surgery (radical prostatectomy) or radical radiotherapy (external beam or brachytherapy). However, approximately 40% of men who have had apparently curative treatment for localised disease will subsequently develop metastatic disease. About 70% of men have metastases at some time during the course of their disease.

For men with metastatic disease, medical or surgical castration often produces a remission but inevitably lethal androgen-resistant disease supervenes that is relatively resistant to chemotherapy, as reviewed in Logothetis, C. J. et al., Semin. Oncol. 21:620, 1994. Metastatic prostate cancer is incurable and the terminal hormone-refractory phase of the disease is least responsive to any kind of treatment.

Surgical castration is achieved through the operation of bilateral orchidectomy and its therapeutic effects are approximately equivalent to complete androgen blockade using a combination of a LHRH agonist/antagonist and an anti-androgen drug (Santen R. J., J Clin. Endocrinal. Metab. 75:685-689, 1992; Thenot, S. et al., Mol. Cell Endocrinol. 156:85-93, 1999). Depending upon the operator, either procedure may be associated with considerable morbidity with incontinence and impotence rates in some series that approach 50%. About 70-80% of men with metastatic disease respond to either kind of hormonal treatment and are palliated effectively for a median duration of approximately two and a half years. Hormonal treatments have side effects of their own, which include lethargy, weakness and cognitive impairment. In time, “androgen-independent” growth of the cancer supervenes, which is usually fatal (Thenot S. et al., supra). This hormone-resistant phase of the disease has a median survival of 40-50 weeks. Combination chemotherapy may produce clinical benefit in approximately 25% of cases but without prolongation of survival.

There is some evidence that cancer patients make spontaneous albeit ineffective immune responses to their own cancers (Lee, P. P. et al., Nature Medicine 5(6):677-685, 1999; Albert, M. L., et al., Nature Medicine 4:1321-1324, 1998). Most of these immune responses are made against normal components of the tissue from which the cancer originates, and are known as differentiation antigens. This has been well demonstrated for melanocyte differentiation antigens, which comprise the major class of defined melanoma tumor antigens (Rosenberg, S. A. et al., Immunity 10:281-287, 1999). Moreover, melanocyte differentiation antigens have been defined as tumor rejection antigens by the adoptive transfer of ex vivo expanded tumor infiltrating lymphocytes (Rosenberg, S. A. et al., J Am Med Assoc 271:903, 1994).

In prostate cancer, on the other hand, the evidence that prostate differentiation antigens are recognized by the immune system of cancer patients is limited. In particular, none of these antigens has been defined as a tumor rejection antigen. However, T cell proliferative responses to human prostate specific antigen (hPSA) and human prostatic acid phosphatase (hPAP) were detected in 6% and 11% of prostate cancer patients, respectively, together with human PAP-specific production of the T helper cytokine, interferon-γ. These findings suggest that an immune environment, which can support PAP-specific cytotoxic T lymphocytes, may exist in prostate cancer patients (McNeel D. G. et al., Cancer Research 61:5161-5167, 2001). Further evidence in support of pre-existing immunity to human PAP, which is T helper cell-dependent, is the discovery of human PAP-specific antibodies in approximately 5% of prostate cancer patients and male controls (McNeel D. G. et al., J. Urinol. 164(5):1825-1839, 2000). Further investigations identified a number of T helper epitopes, which may represent naturally processed human PAP-specific MHC class II epitopes (McNeel et al., 2001, supra). Moreover, antitumor responses were observed in prostate cancer patients who were immunized with dendritic cells loaded either with human prostatic acid phosphatase (hPAP) (Peshwa, M. V. et al., Prostate 36:129-138, 1998) or a peptide derived from human prostate specific membrane antigen (hPSMA) (Lodge, P. A. et al., Cancer Research 60:829-833, 2000; Murphy et al, Prostate 38:73-78, 1999(a); Murphy, G. P. et al., Prostate 39:54-59, 1999(b)).

Considerable effort has consequently been expended in developing therapeutic strategies targeting prostate specific antigens, the most well characterised of these antigens being PSA (Prostate Specific Antigen), PSMA (Prostate Specific Membrane Antigen) and PAP (Prostatic Acid Phosphatase). Prostate cancer is an attractive candidate for immunotherapy because tumors grow slowly and patients have generally been spared immunosuppressive doses of chemoradiotherapy. Fong et al, in particular, have shown anti-PAP T cell proliferative responses in human subjects administered with antigen loaded dendritic cells. Dendritic cells were enriched from peripheral blood mononuclear cells and loaded with mouse PAP to provide xenogeneic stimulation of the immune response (Fong, L. et al., J Immunol 167:7150-7156, 2001).

There is a need, however, for an efficacious, specific and safe immunotherapeutic and/or immunoprophylacetic strategy for the treatment or prevention of prostate cancer. In accordance with the present invention, the inventor has developed such a strategy based on genetic vaccination with a recombinant poxvirus construct expressing a prostate specific polypeptide such as prostatic acid phosphatase preferably together with a immunostimulatory molecule, for example, an immunostimulatory cytokine such as, in particular, IL-2.

### SUMMARY OF THE INVENTION

Throughout this specification, unless the context requires otherwise, the word “comprise”, and variations such as “comprises” and “comprising” will be understood to imply the inclusion of a stated integer or step or group of integers or steps but not the exclusion of any integer or step or group of integers or steps.

Nucleotide and amino acid sequences are referred to by sequence identifier numbers (SEQ ID NO:). The SEQ ID NOs: correspond numerically to the sequence identifiers <400>1, <400>2, etc. A summary of SEQ ID NOS: is provided in Table 1. A sequence listing is provided after the claims.

The present invention provides inter alia a genetic vaccine construct comprising a poxvirus vector which incorporates and, on administration to a subject, expresses in a cell of said subject, a sequence of nucleotides encoding a prostate-specific polypeptide, or a homologue or derivative or analogue thereof, wherein said poxvirus vector does not productively infect said subject.

In some embodiments, the expression products of said genetic vaccine construct stimulate a PAP-specific immune response. In other embodiments, the expression products of said genetic vaccine construct stimulate a prostate cell specific immune response. In other embodiments, expression products of said genetic vaccine construct stimulate autoimmune prostatitis.

Other embodiments of the present invention provide a genetic vaccine construct comprising a poxvirus vector which incorporates and, on administration to a subject, expresses in a cell of said subject, a sequence of nucleotides encoding a prostate-specific polypeptide or a homologue, derivative or analogue thereof and a sequence of nucleotides encoding an immunostimulatory polypeptide, wherein said poxvirus vector does not productively infect said subject.

In some embodiments, the expression products of said genetic vaccine construct stimulate a prostate cell specific immune response. In other embodiments, the expression products of said genetic vaccine construct stimulate autoimmune prostatitis.

Preferred poxvirus vectors are avipox or orthopox vectors. A particularly preferred poxvirus vector is a fowlpox virus vector.

In a related aspect, antibodies, nucleic acid probes and/or other reagents which specifically bind to or are otherwise capable of distinguishing the present genetic vaccine construct or one or more of its expression products are contemplated within the scope of the present invention.

Preferably, the prostate specific polypeptide is a prostatic acid phosphatase, or a homologue, derivative or analogue thereof.

In a further preferred embodiment, the prostatic acid phosphatase is a xenogeneic homologue thereof. In some embodiments xenogeneic homologues for use in human subjects are rodent and more particularly a rat homologue. In particular, rat prostatic acid phosphatase is preferred.

Accordingly, other embodiments of the present invention contemplate a genetic vaccine construct comprising a poxvirus vector which incorporates and, on administration to a subject, expresses in a cell of said subject, a sequence of nucleotides encoding a xenogeneic prostatic acid phosphatase and a sequence of nucleotides encoding an immunostimulatory polypeptide, wherein said poxvirus vector does not productively infect said subject and wherein an expression product of said genetic vaccine construct stimulates a prostate cell specific immune response.

In still other embodiments of the present invention, the immunostimulatory polypeptide is an immunostimulatory cytokine. For example, said cytokine is preferably a Th-1 or Th-2 type cytokine.

Suitable cytokines are one or more of IFNγ, IL-12, IL-2, TNFα, IL-4, IL-7, GM-CSF, IL-6, IL-15, IL-18 or flt-3 ligand.

In preferred embodiments the cytokines are one or more of IL-2, IFNγ or IL-12.

A particularly preferred cytokine is IL-2.

Accordingly, yet other embodiments of the present invention contemplate a genetic vaccine construct comprising a fowlpox virus vector which incorporates and, on administration to a subject, expresses in a cell of said subject, a sequence of nucleotides encoding a xenogeneic prostatic acid phosphatase and a sequence of nucleotides encoding an IL-2 polypeptide, wherein said fowlpox virus vector does not productively infect said subject and wherein an expression product of said genetic vaccine construct stimulates a prostate cell specific immune response.

Still other embodiment of the present invention contemplate a genetic vaccine construct comprising a fowlpox virus vector which incorporates and, on administration to a subject, expresses in a cell of said subject, a sequence of nucleotides encoding a rat prostatic acid phosphatase and a sequence of nucleotides encoding an IL-2 polypeptide, wherein said fowlpox virus vector does not productively infect said subject and wherein an expression product of said genetic vaccine construct stimulates a prostate cell specific immune response.

Suitably, the prostate cell specific immune response comprises proliferation of T cells which enhance inhibition, lysis, or other forms of downregulation of the number or proliferation of prostate derived cells in a subject.

Other embodiments of the present invention provide a composition comprising a genetic vaccine construct comprising a poxvirus vector which incorporates and, on administration to a subject, expresses in a cell of said subject, a sequence of nucleotides encoding a prostate-specific polypeptide, or a homologue or derivative or analogue thereof, together with one or more pharmaceutically acceptable carriers, wherein said poxvirus vector does not productively infect said subject and wherein expression products of said genetic vaccine construct stimulate a prostate cell specific immune response.

Still other embodiments of the present invention provide a composition comprising a genetic vaccine construct comprising a poxvirus vector which incorporates and, on administration to a subject, expresses in a cell of said subject, a sequence of nucleotides encoding a prostate-specific polypeptide, or a homologue or derivative or analogue thereof, and a sequence of nucleotides encoding an immunostimulatory polypeptide, and one or more pharmaceutically acceptable carriers, wherein said poxvirus vector does not productively infect said subject and wherein expression products of said genetic vaccine construct stimulate a prostate cell specific immune response.

Yet other embodiments of the instant invention provide a method for stimulating or otherwise enhancing a prostate cell specific immune response in a subject comprising administration to said subject of an effective amount of a composition comprising a genetic vaccine construct comprising a poxvirus vector which incorporates and, on administration to a subject, expresses in a cell of said subject, a sequence of nucleotides encoding a prostate-specific polypeptide, or a homologue, derivative or analogue thereof, for a time and under conditions sufficient for expression products of said genetic vaccine construct to stimulate or otherwise enhance a prostate cell specific immune response, and wherein said poxvirus vector does not productively infect said subject.

Still another related aspect of the present invention provides a method for immunotherapy and/or immunoprophylaxis of prostate cancer comprising administration of an effective amount of a composition comprising a genetic vaccine construct comprising a poxvirus vector which incorporates and, on administration to a subject, expresses in a cell of said subject, a sequence of nucleotides encoding a prostate specific polypeptide, or homologue, derivative or analogue thereof, wherein said poxvirus vector does not productively infect said subject, and wherein expression products of said poxvirus vector stimulate a prostate cell specific immune response effective in the treatment and/or prophylaxis of prostate cancer.

A further related aspect of the present invention contemplates the use of a genetic vaccine construct in the manufacture of a medicament for the immunotherapy and/or immunoprophylaxis of prostate cancer, wherein said construct comprises a poxvirus vector which incorporates and, on administration to a subject, expresses in a cell of said subject, a sequence of nucleotides encoding a prostate-specific polypeptide, or a homologue, derivative or analogue thereof wherein said poxvirus vector does not productively infect said subject, and wherein said expression products of said poxvirus vector stimulate a prostate cell specific immune response effective in the treatment or prophylaxis of prostate cancer.

Still further embodiments of the present invention contemplate the use of a genetic vaccine construct in the manufacture of a medicament for the immunotherapy and/or immunoprophylaxis of prostate cancer, wherein said construct comprises a poxvirus vector which incorporates and, on administration to a subject, expresses in a cell of said subject, a sequence of nucleotides encoding a prostate-specific polypeptide, or a homologue, derivative or analogue thereof, and a sequence of nucleotides encoding an immunostimulatory polypeptide, wherein said poxvirus vector does not productively infect said subject, and wherein said expression products of said poxvirus vector stimulate a prostate cell specific immune response effective in the treatment or prophylaxis of prostate cancer.

In some embodiments, the prostate-specific polypeptide is a prostatic acid phosphatase or a homologue or derivative or analogue thereof.

A particularly preferred immunostimulatory polypeptide in this embodiment of the invention is an immunostimulatory cytokine. For example, said cytokine is preferably a Th-1 or Th-2 type cytokine.

Preferred examples of cytokine is one or more of IFNγ, IL-12, IL-2, TNFα, IL-4, IL-7, GM-CSF, IL-6, IL-15, IL-18 or flt-3 ligand.

More preferably, the cytokines are one or more of IL-2, IFNγ or IL-12.

A particularly preferred cytokine is IL-2.

### BRIEF DESCRIPTION OF THE DRAWINGS

FIG. 1 is a photographic representation of electrophoretically resolved PCR-fragments. VIR501 and VIR502 were subjected to PCR to test for correct gene insertion. PCR amplifications using human PAP, rat PAP and human IL-2 specific primers for the detection of human IL-2 and rat PAP insertion into VIR501 and human IL-2 and human PAP insertion in VIR502. Negative control experiments where carried out using water instead of DNA template.

FIG. 2 is a photographic representation of ELISA wells showing the results of tests for secretion of human IL-2 by VIR501 and VIR502. After three rounds of plaque purification a number of plaque clones were amplified by infecting CEF cells. After infection, a small sample of culture medium was removed and tested for the presence of human IL-2 using a human IL-2-ELISA kit. A visible colour change indicates the presence of IL-2 in the test sample.

FIG. 3 is a photographic representation of a Western Blot showing FPV-mediated expression of human PAP. TK⁻143B cells were infected with FPV vectors at MOI of 10 and the cells harvested for immunoblot analysis 48 h and 72 h post-infection (p.i.). FPV-HA was used as a negative vector control. The lack of cross-reactivity of the anti-human PAP polyclonal antibody was demonstrated by the absence of a band for recombinant rat PAP. The negative control for the method of detection was probing of the immunoblot with the secondary antibody alone. 1, FPV-HA; 2, VIR501 (48 h p.i.); 3, VIR502 (48 h p.i.); 4, VIR501 (72 h p.i.); 5, VIR502 (72 h p.i.); 6, bacterial recombinant rat PAP (pQE system); 7, molecular weight marker; 8, FPV-HA; 9, VIR501 (48 h p.i.); 10, VIR502 (48 h p.i.). The 49.1 kDa marker together with the location of the specific human PAP bands is indicated.

FIG. 4 is a graphical representation of results of ELISA showing immunogenicity of FPV expressing human PAP. Three NZ White rabbits were immunised by intramuscular injection with 2×10⁸ plaque forming units (PFU) each of empty vector control, FPV-M3, or VIR501 (FPV encoding rat PAP and human IL-2) or VIR502 (FPV encoding human PAP and human IL-2). The animals were boosted according to the same schedule three weeks after the priming immunisation. Two weeks after the boost immunisation the animals were bled again. Reactivities of the immune sera were tested against bacterial recombinant proteins that were made in the pQE system: A. human PAP, or B. rat PAP. Shown are dilution curves for pre-immune sera (light-grey dashed line); post-immune sera, three weeks after priming immunisation (medium grey line with filled triangles); post-immune sera, two weeks after boost immunisation (thick black line with filled diamonds). Negative controls included plates coated with irrelevant pQE-derived proteins, human La/SS-B autoantigen or staphylococcal exotoxin B (SEB) for which OD (at 405 nm) values <0.1 were obtained (data not shown).

FIG. 5 is a representation of the nucleotide sequence of the insertion site of VIR501 containing human IL-2 and rat PAP sequences.

FIG. 6 is a representation of the nucleotide sequence of the insertion site of VIR502 containing human IL-2 and human PAP sequences.

FIG. 7 is a representation of the aligned amino acid sequences of rat PAP from VIR501 with human PAP from VIR502 as indicated.

FIG. 8 is a graphical representation of ELISA showing immunogenicity of FPV expressing rat PAP. Two wethers (castrated male sheep) were immunised by intramuscular injection with either 2×10⁸ plaque forming units (PFU) of VIR501 (FPV encoding rat PAP and human IL-2) [upper panels] or 3×10⁸ PFU of VIR502 (FPV encoding human PAP and human IL-2) [lower panels]. Four weeks after immunisation, sera were collected for ELISA. The reactivity of the immune sera was tested against recombinant proteins that were made either in the pQE bacterial expression system: A. human PAP, B. rat PAP, or the InsectSelect expression system: C. rat PAP (upper panel) or human PAP (lower panel). Shown are dilution curves for pre-immune sera (light-grey dashed line) and post-immune sera, four weeks after the priming immunisation (medium grey line with filled triangles).

FIG. 9 is a schematic representation showing, for VIR501 and VIR502, where the single insertion of human IL-2 and PAP sequences is located in the fowlpox virus genome. The fowlpox virus genome used to construct VIR501 and VIR502 has not yet been sequenced so the fowlpox virus Genbank sequence (AF198100) has been used as a reference for location of insertion site relative to the thymidine kinase (FPV086R)ORF.

FIG. 10 is a schematic representation showing a map of the plasmid integration vector pVHL04, used to construct VIR501.

FIG. 11 is a schematic representation showing a map of the plasmid integration vector pVHL05, used to construct VIR502.

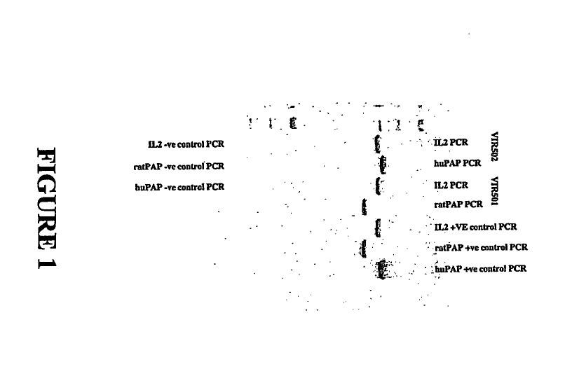

### DETAILED DESCRIPTION OF THE PREFERRED EMBODIMENTS

The present invention is predicated, in part, on the determination that a genetic vaccine construct based on a live poxviral vector which incorporates and expresses in a cell of a subject a sequence of nucleotides encoding a polypeptide which is normally only expressed on or near the surface of prostate cells, preferably together with an immunostimulatory polypeptide, is capable of selectively inducing immune prostatitis in a subject.

Accordingly, the present invention provides inter alia a genetic vaccine and methods for treating or preventing prostate related diseases or conditions such as prostate cancer. Without being limited by any particular theory or mode of operation, by using a poxviral vector which does not productively infect the subject, the risk of an on-going viral infection and/or expression of prostate specific polypeptide in a wide range of possibly inappropriate cells is minimised. Furthermore, by using a prostate specific polypeptide which exhibits a low level of similarity to other polypeptides in the subject, the risk of generating an inappropriate immune response is also reduced.

Accordingly one aspect of the present invention contemplates a genetic vaccine construct comprising a poxvirus vector which incorporates and, on administration to a subject, expresses in a cell of said subject, a sequence of nucleotides encoding a prostate-specific polypeptide, or a homologue or derivative or analogue thereof, wherein said poxvirus vector does not productively infect said subject and wherein expression products of said genetic vaccine construct stimulate a prostate cell specific immune response.

By “genetic vaccine construct” is meant a composition comprising a recombinant nucleic acid molecule which, for the purpose of immunisation is administered to a subject in whom one or more antigenic polypeptides, encoded by at least a part of said nucleic acid molecule, are expressed.

In accordance with the present invention, the poxvirus vector does not “productively infect” the subject. The phrase “does not productively infect” or no “productive infection” means that the vector may infect cells of the subject, for instance near the zone of administration, however, the virus does not propagate and the risk of an on-going viral infection and/or prostate specific polypeptide expression in a wide range of possibly inappropriate cells is minimised. In particular this may occur because viral replication is inefficient, incomplete, or limited.

For example, it would be inappropriate or undesirable for the poxviral vector to propagate and spread in cells of important non-prostate cell organs thus making them targets of immune destruction. Of course, initial infection and expression of proteins by the vector is required and engenders an immune response.

Those skilled in the art will know that poxviruses comprise a diverse group of viruses classified traditionally according to their host range. For example, wild type avipox viruses do not replicate in the cells of non-avian species. The limiting step in replication is inefficient late gene expression or inefficient maturation of viral particles (Somogyi P. et al., Virology 197:439-444, 1993). However, genes under the control of early poxviral promoters are expressed in the cells of non-avian species such as man and heterologous genes are routinely expressed in this way (Taylor, J. et al., Vaccine 6:497-503, 1988; Cox, W. et al., Virology 195:845-850, 1993). In immunocompetent hosts, some poxviral infections such as, for example, infection with certain strains of vaccinia in man are generally limited, nevertheless, man is a host species for vaccinia virus and at least initially, substantial viral replication would be expected.

The measure for absence of productive infection in accordance with the present invention is the inability of the poxvirus vector to propagate in or spread from the cells initially infected. In a preferred embodiment, the absence of productive infection in a subject is less than approximately 10% of total viral replication observed in a permissive host, preferably less than 5%, more preferably less than 1%, even more preferably less than 0.1% and still more preferably less than 0.01%. The choice of poxvirus vector will therefore depend, inter alia, on the subject species.

For the avoidance of any doubt, in accordance with the present invention, the recombinant avipox including fowlpox vectors of the present invention do not productively infect non-avian hosts.

Alternatively, a conditionally replication defective poxvirus vector may be engineered by methods known in the art not to productively infect a host. For example, some aspects of the genetic basis for host specificity in vaccinia poxvirus strains are understood and replication defective vaccinia viruses have been generated by deletion of “host range” genes (Perkus, M. E. et al., Virology 179:276-28, 1990). Also replication deficient or attenuated viruses such as modified vaccine virus (MVA) are examples of poxviruses which do not productively infect a human subject. Such modified or attenuated poxvirus vectors may be obtained by repeated passage of viruses in cells in vitro, for example in chicken embryo fibroblasts.

Reference to “poxvirus” includes viruses selected from, for example, avipox (eg, fowlpox, canarypox, penguinpox, pigeonpox) orthopox (eg, vaccinia) capripox (eg, sheep, goats) and suipox (eg, swinepox). Avipox vectors are preferred vectors. A particularly preferred vector is fowlpox.

Although human subjects are primarily contemplated, reference to a “subject” should be understood to include mammals including primates (eg, humans, monkeys), livestock animals (eg, sheep, cows, horses, donkeys, goats, pigs), laboratory test animals (eg, mice, rats, ducks, dogs, guinea pigs, rabbits, hampsters), companion animals (eg, dogs, cats, birds), and captive wild animals (eg, kangaroos, deer, foxes). Preferably said subject is a primate and even more preferably a human subject.

Reference to a “cell” in “expresses in a cell” includes expression in antigen presenting cells such as dendritic cells.

The general principles and procedures for generating and using recombinant poxvirus vectors are well known in the art. Briefly, homologous recombination between a donor recombination vector and a poxvirus within a host cell permits correct introduction of the desired sequences. The donor vector comprises a sequence of nucleotides permitting site specific homologous recombination with a poxvirus vector, a sequence of nucleotides encoding a prostate specific polypeptide together with one or more of any other elements required for amplification in a prokaryotic host, selection of transfected cells, and transcription of nucleic acid sequences. Double and further recombinants such as a vector further comprising a sequence of nucleotides encoding an immunostimulatory polypeptide or peptide are generated in essentially the same manner, however different promoters and selection markers may be employed.

In a preferred aspect, the present invention contemplates a genetic vaccine construct comprising an avipox vector which incorporates and, on administration to a subject expresses in a cell of said subject, a sequence of nucleotides encoding a prostate-specific polypeptide, or a homologue or derivative or analogue thereof, wherein said avipox vector does not productively infect said subject and wherein expression products of said genetic vaccine construct stimulate a prostate cell specific immune response.

A fowlpox vector is a preferred avipox vector. Fowlpox viruses are preferred inter alia because, they express appropriate levels of heterologous protein. The use of fowlpox virus in man may also be preferred because immunity to fowlpox would not generally be present. In contrast, a large proportion of the human population has been exposed to vaccinia virus as a result of prior vaccination regimes. As a result, the introduction of vaccinia virus into a human patient can provoke an immune response to the vaccinia viral vector. In this situation, the vector may be neutralised before any antigenic proteins are expressed.

The genetic vaccine construct of the present invention may also comprise a sequence of nucleotides which is a useful marker for detection by nucleic acid based assays, or expressed and useful for detection such as by protein assays including enzyme or antibody based assays.

The present vectors may be identified using any suitable protocol such as heteroduplex analysis, polymerase chain reaction (PCR), ligase chain reaction (LCR), sequence specific hybridization probes (SSO), single-stranded conformational polymorphism (SSCP), sequencing, mass spectrometry, enzyme cleavage, protein probes including antibody, enzyme or immunoreactive based assays and combinations of these.

Another aspect of the present invention contemplates an isolated antibody which is determined by epitopes which are uniquely formed in expression products of the subject genetic vaccine construct.

Isolated antibodies may be monoclonal or polyclonal. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies.

In one embodiment, specific antibodies can be used to screen a sample from a subject for the presence of expression products of the vaccine construct.

Alternatively, the ability of a subject to mount a specific antibody response to a proteinaceous form of the vaccine construct may be used to determine whether a subject has previously been vaccinated with the subject vaccine construct. Techniques for the assays contemplated herein are known in the art and include, for example, sandwich assays and ELISA.

Both polyclonal and monoclonal antibodies are obtainable by immunization with the enzyme or protein and either type is useful for immunoassays. The methods of obtaining both types of sera are well known in the art. Polyclonal sera are less preferred but are relatively easily prepared by injection of a suitable laboratory animal with an effective amount of a proteinaceous form of a molecular marker, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting a proteinaceous form of the instant genetic poxviral vaccine construct in a subject said method comprising contacting a biological sample from said subject with an antibody specific for a proteinaceous form of the genetic poxviral vaccine construct for a time and under conditions sufficient for an antibody-antigen complex to form, and then detecting said complex.

The presence of a complex may be detected in a number of ways such as by Western blotting and ELISA procedures. A wide range of immunoassay techniques are available as can be seen by reference to U.S. Pat. Nos. 4,016,043, 4,424,279 and 4,018,653. These includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent.

The sample is generally a biological sample comprising biological fluid but also includes to supernatant fluid such as from a cell culture. Methods of sample preparation are well known to those skilled in the art.

“Reporter molecule” as used in the present specification, means a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength. The fluorescence observed indicates the presence of the molecule of interest. Immunofluorescence and EIA techniques are both very well established in the art and are particularly preferred for the present method. However, other reporter molecules, such as radioisotope, chemiluminescent or bioluminescent molecules, may also be employed.

The phrase “expression products” includes the products of transcription and/or translation. Accordingly proteins are preferred products but the activity of transcripts in RNA form is not excluded from the scope of the present invention.

The phrase “prostate specific polypeptide” is used in a broad sense and includes a polypeptide which is expressed on or near the surface of prostate cells, including prostate cancer cells, and is not substantially expressed on the surface of non-prostate cells. In this way, an immune response is directed specifically to prostate cells and not other self cells of the subject.

A preferred prostate specific polypeptide is a polypeptide which has a low level of similarity to other polypeptides in a subject. This aspect provides that an immune response is directed preferentially to prostate cells expressing a prostate specific polypeptide and rather than cells expressing a cross reactive epitope not determined by a prostate specific polypeptide.

By way of further clarification a prostate specific polypeptide is not substantially expressed on or near the surface of non-prostate cells if it is expressed at a level of less than about 10% of that determined on or near the surface of prostate cells and more preferably less than 5%, more preferably less than 1%, even more preferably less than 0.1%, even still more preferably less than 0.01% or even yet still more preferably less than 0.001%.

In a particularly preferred embodiment, the subject prostate specific polypeptide is a prostatic acid phosphatase. Advantageously, prostatic acid phosphatase (PAP) is expressed specifically in the prostate cells including prostate cancer cells and it has been used widely as a marker for prostate cancer. Additionally PAP exhibits a low level of amino acid and nucleotide sequence similarity to known proteins and their encoding nucleic acids. PAP also has a range of homologues which exhibit a high level of amino acid and nucleotide sequence similarity.

Homologues, derivatives or analogues of prostate specific polypeptides and their encoding nucleotide sequences are clearly contemplated. Generally, such forms exhibit comparable or enhanced function in the present invention, relative to sequences from which they are derived or based.

For the purpose of the present invention a derivative of the subject nucleic acid sequences may be a functional part or fragment which achieves the advantage of the present invention or it may comprise one or more mutations or modifications. Mutations include one or more nucleotide deletions, insertions or substitutions. Mutations may be silent, conservative, missense or frameshift mutants provided that the antigenic function of the polypeptide expressed therefrom is retained or enhanced.

Preferably, derivatives have at least 50% similarity to the pre-derivatised or parent molecule, preferably at least, 60%, 65%, 70%, 75%, 80%, 85%, 90%, 95%, 99% similarity to the pre-derivatised or parent molecule. Sequence comparisons are preferably the whole molecule but may also be part thereof, preferably the comparisons are made in a continuous series of at least about 21 nucleotides. The nucleotide sequences of prostate specific polypeptides such as PAP, PSMA and PAP and are published in Genbank. Homologues from other species are readily obtained by well known screening and cloning methods.

Functional derivatives may be obtained by any route and may be synthetic or recombinant. A straightforward but random route is to use mutagenesis followed by testing or expression and testing of the expression products such as by testing its ability to induce anti-polypeptide immune response. Additionally, derivatives may be modified to have other useful properties such as to enhance processing and presentation of the expressed peptide in order to enhance the immune response thereto. Alternatively, or in addition, derivatives may maintain function whilst having additional features such as modifications which permit the polypeptide or peptide to be distinguished over the wild type polypeptide.

Analogues are not a part or mutant form of the parent molecule but they have an analogous function. Analogues may be recombinant or synthetic and preferably have enhanced function over the parent molecule for instance by excluding immunosuppressive epitopes. Analogues may be designed so that their expressed proteins mimic certain immunological or physiochemical property of the prostate specific polypeptide.

Homologues of prostate specific polypeptides include isoenzymes, splice variants, tissue specific forms and species specific forms of the polypeptide. Species homologues are also referred to as xenogeneic forms of prostatic acid phosphatase and include, of course, primate, mammalian and rodent homologues. By “xenogeneic” is meant the use of forms derived from a different species compared to the species' origin of the subject. Thus, for human subjects, a xenogeneic prostate specific polypeptide is any form wherein it is not derived from humans. Preferably, homologues exhibit a high level of sequence or immunological similarity. Derivatives and analogues of the instant homologues are also contemplated herein.

Usefully, various algorithms are available in the art that permit analysis of peptide sequences and homologues thereof to determine the likelihood that they will exhibit enhanced function. For example, the Parker algorithm (Parker, K. C. et al., Journal of Immunology 152:163-175, 1994) estimates half-times of dissociation for MHC class-I peptide binding motifs.

Similarity at the nucleic acid level may be assessed in assays exploiting different hybridisation conditions as is well known in the art and is, for example, described in Ausubel et al., 2002. Preferably, a derivative nucleic acid molecule of the invention is capable of hybridizing to a reverse complement of a nucleotide sequence encoding a prostate specific polypeptide under low stringency conditions at 42° C., more preferably under medium stringency and most preferably under high stringency conditions.

Low stringency hybridisation conditions includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions.

Medium stringency includes and encompasses from at least about 16% v/v to at least about 30% v/v formamide and from at least about 0.5 M to at least about 0.9 M salt for hybridization, and at least about 0.5 M to at least about 0.9 M salt for washing conditions. High stringency includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur et al., J. Mol. Biol. 5:109, 1962). However, the Tₘ of a duplex DNA decreases by 1° C. with every increase of 1% in the number of mismatch base pairs (Bonner et al., Eur. J. Biochem. 46(1): 83-88, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels.

Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. A detailed discussion of sequence analysis can be found in Unit 19.3 of Ausubel et al.

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

In accordance with one aspect of the present invention, the prostate specific polypeptide has no more than about 70% amino acid similarity to other antigenic proteins in the subject. More preferably, the prostate specific polypeptide has no more than 60%, even more preferably no more than about 50% amino acid similarity.

Another aspect of the present invention consequently provides a genetic vaccine construct comprising an avipox vector which incorporates and, on administration to a subject expresses in a cell of said subject, a sequence of nucleotides encoding a prostatic acid phosphatase, and/or a homologue or derivative or analogue thereof, wherein said avipox vector does not productively infect said subject and wherein expression products of said genetic vaccine construct stimulate a prostate cell specific immune response.

In a particular embodiment, a xenogeneic homologue of the prostatic acid phosphatase is a preferred homologues which exhibits potentially higher binding affinity for more HLA molecules than an indigenous prostatic acid phosphatase homologue.

In accordance with one aspect of the present invention the inventor has determined that rat PAP-derived motifs exhibit higher binding affinities for more HLA molecules than human PAP-derived motifs. Accordingly xenogeneic administration is proposed for some applications. A preferred xenogeneic form of prostatic acid phosphatase for particular use in human subjects is rat PAP. Without intending to be limited by any one particular mechanism or mode of action, the use of a xenogenic homologue is provided to assist in overcoming self tolerance and to illicit effective effector cells such as, for example, CD4⁺ and CD8⁺ T cells and Natural Killer (NK). Effective T cells are generally high affinity and/or high avidity immune effector cells. Combinations of xenogeneic and indigenous prostate specific polypeptides are also contemplated.

Another aspect of the present invention provides a genetic vaccine construct comprising a poxvirus vector which incorporates and, on administration to a subject expresses in a cell of said subject, a sequence of nucleotides encoding a xenogeneic homologue of prostatic acid phosphatase, or a further derivative or an analogue thereof, wherein said poxvirus vector does not productively infect said subject and wherein expression products of said genetic vaccine construct stimulate a prostate cell specific immune response.

The phrase “stimulates a prostate cell specific immune response” includes reference to inducing or enhancing or otherwise stimulating a cellular and/or humoral immune response in a subject to one or more antigenic components of a prostate specific polypeptide which is expressed on or near the surface of prostate cells including prostate cancer cells. In a preferred embodiment, the immune response comprises cellular and humoral responses sufficient to generate immune prostatitis including antigen specific cytotoxic cells which inhibit, lyse or otherwise down regulate the number or proliferation of prostate cells including prostate cancer cells, if present, in a subject. Even more preferably, the immune response is directed selectively towards prostate cells including prostate cancer cells, if present, and is not directed to other cells in the subject. As previously canvassed, in exploiting self antigens in vaccines, the present inventor has selected polypeptides which are essentially specific to the prostate and which furthermore exhibit a low level of amino acid or nucleotide sequence similarity to other proteins in the subject.

Various algorithms and assays including in vitro and in vivo assays are available to test or predict the effectiveness and/or suitability of particular genetic vaccine constructs within the scope of the present invention. In particular, various cellular and animal models of prostate cancer in humans are available including primate, dog and rodent models.

In a further related embodiment the prostate cell specific immune response is enhanced by co-expressing the prostate specific polypeptide with an immunostimulatory molecule.

By “enhanced” is meant that administration of the present composition results in a prostate cell specific immune response which is more effective in treating or preventing prostate-related diseases or conditions in a subject than the immune response, if any, in that subject prior to administration of the present composition.

Accordingly, another aspect of the present invention provides a genetic vaccine construct comprising a poxvirus vector which incorporates and, on administration to a subject expresses in a cell of said subject, a sequence of nucleotides encoding a prostate-specific polypeptide or a homologue, derivative or analogue thereof and a sequence of nucleotides encoding an immunostimulatory molecule, wherein said poxvirus vector does not productively infect said subject and wherein expression products of said genetic vaccine construct stimulate a prostate cell specific immune response.

The term “immunostimulatory molecule” is used in its broadest sense and includes polypeptides or functional parts thereof which stimulate or enhance a prostate cell specific immune response generated by the immune system in response to the herein described genetic vaccine construct. The immunostimulatory molecule may, in the case of particular prostate specific polypeptides or particular poxviral vectors described herein, be required in order to generate immune prostatitis. In other embodiments, the immunostimulatory molecule modulates and/or enhances the immune response.

Preferred immunostimulatory polypeptides include all or a functional part of polypeptides including cytokines, chaperokines, chemokines, accessory or adhesion molecules such as B7 and ICAM. Polypeptides which down regulate immunoinhibitory molecules are also encompassed by the present invention.

In a preferred embodiment the immunostimulatory molecule is a cytokine. In accordance with the present invention it is contemplated that the cytokine is co-expressed with one or more prostate specific polypeptides. During antigen processing, the cytokine modulates the immune response to enhance its effectiveness. Preferred cytokines are one or more of IFNγ, IL-12, IL-2, TNFα, IL-4, IL-7, GM-CSF or IL-6. Even more preferred cytokines are one or more of IL-2, IFNγ or IL-12. A particularly preferred cytokine is IL-2.

IL-2 is a preferred cytokine, inter alia, because of its ability to enhance the immune response to the instant vector and because of its documented safety in humans under controlled conditions. In treating human subjects, human-derived cytokines are preferred.

Accordingly, yet another aspect of the present invention contemplates a genetic vaccine construct comprising a fowlpox virus vector which incorporates and, on administration to a subject, expresses in a cell of said subject, a sequence of nucleotides encoding a xenogeneic prostatic acid phosphatase and a sequence of nucleotides encoding an IL-2 polypeptide, wherein said fowlpox virus vector does not productively infect said subject and wherein expression products of said genetic vaccine construct stimulates a prostate cell specific immune response.

Still another aspect of the present invention contemplates a genetic vaccine construct comprising a fowlpox virus vector which incorporates and, on administration to a subject, expresses in a cell of said subject, a sequence of nucleotides encoding a rat prostatic acid phosphatase and a sequence of nucleotides encoding an IL-2 polypeptide, wherein said fowlpox virus vector does not productively infect said subject and wherein expression products of said genetic vaccine construct stimulates a prostate cell specific immune response.

Yet another related aspect of the instant invention provides a method of stimulating or otherwise enhancing a prostate cell specific immune response in a subject comprising administration to said subject of an effective amount of a composition comprising a genetic vaccine construct comprising a poxvirus vector which incorporates and, on administration to a subject expresses in a cell of said subject, a sequence of nucleotides encoding a prostate-specific polypeptide, or a homologue, derivative or analogue thereof, for a time and under conditions sufficient to stimulate or otherwise enhance a prostate cell specific immune response, and wherein said poxvirus vector does not productively infect said subject.

Administration of the genetic vaccine construct composition may be optimised using protocols which are well known in the art. In particular the dose and frequency will vary with the mode of administration as well as various parameters relating to the subject including size, previous exposure to the vaccine, stage of prostate cancer development. The composition may be administered by any convenient route such as by oral, intravenous, intranasal, intramuscular, intraperitoneal, subcutaneous, intradermal, mucosal or suppository routes. Preferred modes of administration are intravenous or intramuscular, however, the chosen route will be influenced by factors such as cost and the stability of the dosage form.

An “effective amount” includes reference to a virus titre necessary to at least partly obtain the desired immune response overall. This will of course vary with the status of the subject and accordingly is optimised during pre-clinical and clinical investigations.

Various adjuvants may be used to enhance the efficacy of the subject vaccine. Examples include alum, lecithins, BCG and saponins, or cellular adjuvants such as dendritic cells.

The vaccine composition may be co-administered or administered as part of an overall vaccination regime, with other molecules. For example, the subject vaccine constructs and its expression products may be administered as part of a prime or boost vaccination component in a “prime-boost” strategy wherein the immune response is enhanced by presenting antigens to the immune system via various formats.

Still another related aspect of the present invention provides a method of immunotherapy and/or immunoprophylaxis of prostate cancer comprising administration of an effective amount of a composition comprising poxvirus vector which incorporates and, on administration to a subject, expresses in a cell of said subject, a sequence of nucleotides encoding a prostate specific polypeptide or homologue, derivative or analogue thereof, wherein said poxvirus vector does not productively infect said subject, and wherein expression products of said poxvirus vector stimulate a prostate cell specific immune response effective in the treatment and/or prophylaxis of prostate cancer.

Reference to “immunotherapy” includes amelioration of the symptoms of prostate cancer or a reduction in the number or proliferation of prostate cancer cells as well as treatment to total recovery. Reference to “immunoprophylaxis” includes prevention of developing prostate cancer or the symptoms of prostate cancer as well as a reduction in the likelihood of developing the symptoms or more severe symptoms of prostate cancer. If a subject were diagnosed as exhibiting a marker for progression or a marker for susceptibility to prostate cancer, the poxvirus vector is administered before diagnosis of prostate cancer.

Pharmaceutical forms of the composition may be suitable for injectable use such as sterile aqueous solutions and sterile powders for the extemporaneous preparation of sterile injectable solutions or dispersions.

The composition must be stable under the conditions of manufacture and storage and must be preserved against the contaminating action of microorganisms such as bacteria and fungi. The carrier can be a medium solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the viral particles in the required amount in the appropriate medium with optionally various of the other ingredients enumerated above, as required. Batches are tested for sterility contamination with protein, virus concentrate pfu/ml) virus stability, pH and fill volume.

A broad range of doses may be applicable depending on the subject, severity of condition and proposed route and medium for administration.

It is especially advantageous to formulate parenteral compositions in dosage unit form (pfu/ml) for ease of administration and uniformity of dosage. Dosage unit form as used herein refers to physically discrete units suited as unitary dosages for subjects to be treated; each unit containing a predetermined quantity of active material calculated to produce the desired therapeutic or prophylacetic effect in association with a pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail. Techniques for enterically locating live vaccine formulations are known in the art.

A further related aspect of the present invention contemplates the use of a genetic vaccine construct in the manufacture of a medicament for the immunotherapy and/or immunoprophylaxis of prostate cancer, wherein said construct comprises a poxvirus vector which incorporates and, on administration to a subject expresses in a cell of said subject, a sequence of nucleotides encoding a prostate-specific polypeptide, or a homologue, derivative or analogue thereof wherein said poxvirus vector does not productively infect said subject and wherein expression products of said poxvirus vectors stimulate a prostate cell specific immune response effective in the treatment or prophylaxis of prostate cancer.

A still further related aspect of the present invention contemplates the use of a genetic vaccine construct in the manufacture of a medicament for the immunotherapy and/or immunoprophylaxis of prostate cancer, wherein said construct comprises a poxvirus vector which incorporates and, on administration to a subject expresses in a cell of said subject, a sequence of nucleotides encoding a prostate-specific polypeptide, or a homologue, derivative or analogue thereof and a sequence of nucleotides encoding an immunostimulatory polypeptide, wherein said poxvirus vector does not productively infect said subject and wherein expression products of said poxvirus vectors stimulate a prostate cell specific immune response effective in the treatment or prophylaxis of prostate cancer.

In a related aspect of this embodiment, the prostate-specific polypeptide is prostatic acid phosphatase and/or a homologue or derivative or analogue thereof.

A particularly preferred immunostimulatory molecule for this embodiment of the invention is an immunostimulatory cytokine such as, for example, a cytokine selected from one or more of IFNγ, IL-12, IL-2, TNFα, IL-4, IL-7, GM-CSF or IL-6. Even more preferred cytokines are one or more of IL-2, IFNγ or IL-12. A particularly preferred cytokine is IL-2.

The present invention further provides a genetic vaccine construct as described herein for use in therapy. The present invention additionally provides use of a genetic vaccine construct as described herein in the manufacture of a medicament for treatment or prophylaxis of prostate cancer.

The present invention is now further described with reference to the following non-limiting Examples.

TABLE 1

| SEQ ID NO. | SEQUENCE |
|---|---|
| SEQ ID NO: 1 | Nucleotide sequence of coding strand of |
| | insertion site for VIR501 |
| SEQ ID NO: 2 | Non-coding strand for SEQ ID NO: 1 |
| SEQ ID NO: 3 | Nucleotide sequence of coding strand of |
| | insertion site for VIR502 |
| SEQ ID NO: 4 | Non-coding strand for SEQ ID NO: 3 |
| SEQ ID NO: 5 | Amino acid sequence of rat PAP |
| SEQ ID NO: 6 | Amino acid sequence of human PAP |
| SEQ ID NO: 7 | Amino acid sequence of IL-2 |
| SEQ ID NO: 8 | Rat PAP PCR primer |
| SEQ ID NO: 9 | Rat PAP PCR primer |
| SEQ ID NO: 10 | Human PAP PCR primer |
| SEQ ID NO: 11 | Human PAP PCR primer |

### EXAMPLE 1

### Construction of a Genetic Vector Comprising a Prostate Specific Polypeptide

Human and rat PAP nucleic acid sequences are publicly available, and their cDNAs may be cloned and sequenced using routine methods well known to those skilled in the art. Bacterial recombinant rat PAP and human PAP plasmid vectors were obtained from Dr Doug McNeel (Department of Medicine, division of Medical Oncology, University of Washington, Seattle, Wash. 98195, USA) and their products are used for coating ELISA plates. Recombinant rat PAP and human PAP proteins have been made in the InsectSelect system and scaled-up production of purified proteins is done so that they may be used in both rat and human cellular immunological assays.

The recombinant fowlpox viruses expressing human PAP (FPV.hPAP) and rat PAP (FPV.rPAP) can be generated using molecular biology techniques for shuttle vector construction using procedures described by Sambrook et al. “Molecular Cloning: A Laboratory Manual” Cold Spring Harbor Laboratory, 3rd Edition, 2001 and using molecular virology technique to generate recombinant poxviruses using procedures described by Boyle, D. B., et al, Gene 65(1):123-8, 1988; Coupar, B. E. et al., Gene 68(1): 1-10, 1988 and Smith G. L., Chapter 9, Expression of genes by vaccinia virus. In Molecular Virology, A Practical Approach. Ed. A J Davison and R M Elliott. Practical Approach Series, IRL Press at Oxford University Press., 257-283, 1993. Construction of FPV.hPAP and FPV.rPAP is briefly outlined as follows.

i. PAP Expression Cassette

The PAP protein coding sequence, being either human or rat origin, was operatively linked to a fowlpox virus specific promoter sequence. The promoter sequence in this case does not have to be fowlpox virus specific or vaccinia specific and other Avipox derived promoters can be used and can be of any of the following classes: early, late or early/late (constitutive) promoters. A preferred element for efficient early phase expression during an infection is the presence of a poxvirus early transcriptional stop sequence of the motif “TTTTTNT”, where N can be any nucleotide sequence, e.g., A or T or G or C, which must be located 3′ downstream of the PAP translational stop codon. Addition of this early transcriptional stop motif will not be necessary if such a motif occurs by chance some distance down stream of the PAP translational stop codon. This motif can be conveniently added to the PAP sequence by RT-PCR amplification (using RNA as template) or PCR amplification (using cDNA as template) with a primer combination that includes this motif in the PCR primer that targets the 3′ end of the PAP nucleotide sequence.

ii. Homologous Recombination Vector (so Called Shuttle Vector) for Aiding Insertion of the PAP Expression Cassette into the Fowlpox Genome

The expression cassette described above in step (i) was cloned into a plasmid vector termed “shuttle vector” or “homologous recombination vector” resulting in a configuration described as follows.

The PAP expression cassette was cloned in between two short fowlpox nucleotide sequences of determined length that are homologous to nucleotide sequences present in fowlpox genomic DNA that has been previously cloned into a standard commercial bacterial plasmid vector used for cloning purposes. These short fowlpox nucleotide sequences are often referred to as homologous recombination arms (left and right) of flanking arms (flank 1 and flank 2). The key feature here is that the expression cassette is located within (interior of) the two flanking arms and not exterior to these arms. The result of homologous recombination between these arms and their homologous sequence within the fowlpox genome will aid insertion of the expression cassette into the fowlpox genome. Examples of suitable insertion sites include the TK coding region, 3′ of the TK coding region and the ORF7 to ORF9 region (U.S. Pat. No. 5,180,675).

The shuttle vector also contained a “reporter” expression cassette (beta-galactosidase protein coding sequence operatively link to a poxvirus specific promoter) and a “positive selection” expression cassette (E coli xanthine-guanine phosphoribosyl transferase (Ecogpt) operative linked to a poxvirus specific promoter) located exterior to the two homologous recombination arms. This configuration enables “transdominant selection” of recombinant viruses.

iii. Homologous Recombination

The insertion of the PAP expression cassette into the fowlpox virus genome was carried by homologous recombination between the fowlpox virus genomic DNA, present during an infection of tissue culture cells, and the shuttle vector described above in (ii). Chicken embryo derived cells were infected with fowlpox virus at low multiplicity of infection, for example, 0.01 infectious units per cell. An hour or two after infection the shuttle vector containing the PAP expression cassette was transfected into these infected cells using commercially available transfection kits following the suppliers instruction. After transfection, the cells and medium were harvested once the infection had reached confluency. A viral extract was prepared by releasing the virus from the infected cells either by mechanical means or repeated cycles of freezing and thawing or by sonication. Two homologous recombination setups were prepared, one for making a recombinant fowlpox virus expressing the rat PAP and another to make a recombinant fowlpox virus expressing human PAP.

iv. Clone Purification of Recombinant Fowlpox Viruses Expressing PAP

The viral extracts from the homologous recombination step were subjected to multiple rounds of plaque purification in chicken embryo derived cells until no “white” plaques could be observed when Xgal was present in the tissue culture medium. Mycophenolic acid, Xanthine and hypoxanthine (as described in Smith G. L. 1993, supra) was also present in the culture medium during the infection for the purpose of positively selecting recombinant viruses that had a functional Ecogpt inserted into their genomes. The mycophenolic acid will inhibit the replication of non-recombinant virus. This selection procedure will select for viruses where a single recombination between one of the homologous arms and the viral genome inserts the whole shuttle vector into the viral genome as viruses without the Ecogpt will not replicate under this selection environment. Viral clones that produce blue plaques in the presence of Xgal were then amplified with out mycophenolic selection and tested for presence or absence of non-recombinant virus (empty vector) by PCR analysis using PCR primers that target the flanking region of the site of insertion.

Recombinant fowlpox virus clones that were tested negative for empty vector contamination was then subjected to further rounds of plaque purification in the absence of mycophenolic acid, Xanthine and hypoxanthine to encourage the second recombination event that will result in the deletion of the reporter and positive selection cassettes from the recombinant virus. Clones that resulted in white plaques after the addition of Xgal to the culture medium were amplified and tested for empty vector contamination, removal of reporter and positive selection cassettes and for functionality of PAP expression.

A recombinant fowlpox vector (M3) encoding human PAP (FPV.hPAP) is plaque purified and amplified to a titre of 10⁹ pfu/mL. The presence of the human PAP insert is confirmed by PCR. Absence of contaminating wild type fowlpox virus is also confirmed by PCR. Western blot analysis demonstrates the presence of secreted PAP in the supernatant of chicken embryo skin (CES) cells, which were infected with FPV.hPAP.

The FPV.rPAP preparation is plaque-purified twice and the presence of the rat PAP insert confirmed by PCR.

The FPV.rPAP vector is subjected to a third and final round of plaque purification. Then the plaque-purified vector is amplified to high titre. Expression of secreted recombinant rat PAP is assayed by Western blot of FPV.rPAP-infected CES cells. The absence of contaminating wild type FPV is confirmed by PCR. Western blot analysis of human monocyte-derived dendritic cells (moDC), which have been infected in vitro with FPV.rPAP or FPV.hPAP, is done to demonstrate that the fowlpox-vectored transgenes are expressed by the cell type that is most likely to be the target for expression in vivo.

### EXAMPLE 2

### Construction of Genetic Vectors that Co-Express an Immunostimulatory Molecule

The human IL-2 (hIL-2) cDNA has been cloned by RT-PCR from human peripheral blood lymphocytes (PBL), which were activated for 24 h by PMA and ionomycin. The presence of the correct DNA sequence was confirmed by DNA sequence analysis.

Insertion of a Fowlpox Virus Specific Human IL-2 Expression Cassette into FPV.hPAP and FPV.rPAP

The human IL-2 cDNA was operatively linked to a fowlpox virus specific promoter. Alternatives to fowlpox specific promoters can be vaccinia specific promoters of other Avipox virus specific promoters. To this promoter plus hIL2, a poxvirus early transcriptional stop sequence was added downstream of the IL-2 translational stop codon.

This expression cassette was cloned into a fowlpox shuttle vector with the same configurations and features as described in step ii) of Example 1, except that the homologous recombination arms were homologous to a different area of the fowlpox virus genome than used for the PAP shuttle vectors.

Homologous recombination and viral selection were carried out as described in Example 1. The end result was two recombinant fowlpox viruses both expressing human IL-2 but one expressing human PAP (FPV.hPAP/hIL-2) and the other expressing rat PAP (FPV.rPAP/hIL-2). ELISA was used to measure the in vitro production of human IL-2 upon infection of tissue culture cells by any of these two vectors.

### EXAMPLE 3

### In Vivo Immunogenicity of Xenogeneic Genetic Vaccine Viral Construct

The immunogenicity of viral constructs is determined in appropriate animal models and in illustrative embodiments, the immunogenicity of FPV.rPAP and FPV.rPAP/hIL-2 is determined in mice and rabbits.

For detection of anti-rat PAP antibodies, rabbits are immunized with 1×10⁷ pfu FPV.rPAP or FPV.rPAP/hIL-2 IMI then bled 28 d post-immunization for direct ELISA of serum for rat PAP-specific antibodies. Where rat PAP-specific antibodies are not detected at 28 d post-immunization then animals are boosted with FPV.rPAP. As a positive control for both antibody production and the ELISA, rabbits are immunized with recombinant rat PAP in CFA and boosted with recombinant rat PAP in IFA at day 21. Blood is drawn and serum prepared for ELISA 14 days after boosting.

For detection of cellular responses to rat PAP, mice are immunized with 1×10⁷ pfu FPV.rPAP or FPV.rPAP/hEL-2 IMI. Cytolytic and proliferative cellular responses are measured using spleens harvested from mice killed 6 days and 14 days post-immunization, respectively. For detection of rat PAP-specific cytotoxic T lymphocytes (CTL), either intracellular expression of IFNγ or cytolytic function by chromium release assay is measured. Nylon-wool purified splenic T cells are incubated for 6 hours with either irradiated syngeneic antigen presenting cells (APC): EL-4 cells that have been transfected with rat PAP or EL-4 cells as a negative control. Surface staining for CD8 and intracellular staining for IFNγ is assayed by flow cytometry. Alternatively, purified splenic T cells are incubated for 4 hours with ⁵¹Cr-labelled EL-4 cell transfectants or EL-4 cells and antigen-specific chromium release measured. For detection of rat PAP-specific proliferative responses, splenic T cells are purified over a nylon wool column and incubated for 3 days with irradiated syngeneic splenocytes, which have been loaded with recombinant rat PAP or chicken ovalbumin as a negative control. In the final 18 hours of culture, tritiated thymidine will be added and its incorporation measured as an index of antigen-specific proliferation.

### EXAMPLE 4

### In Vitro Immunogenicity of Genetic Vaccine Viral Construct

PAP5 is a HLA-A2.1-binding peptide epitope of human PAP that is identical in rat PAP. Peshwa et al, describe how PAP5-specific CTL can be derived in vitro and propagated as cell lines that lyse both PAP5-loaded T2 cells or the HLA-A2.1⁺ and PAP⁺ prostate cancer cell line, LNCaP. Peripheral blood mononuclear cell (PBMC) cultures from HLA-A2⁺ donors are obtained and stimulated with PAP5 peptide.

PBMC cultures that continue to grow in response to PAP5 peptide are cloned and expanded. PAP5 peptide-specificity is tested by IFNγ-ELISPOT assay. Where clones are positive, they are propagated on PAP5-loaded T2 cells. The cytolytic activity of PAP5-specific CTL is tested in a chromium release assay using LNCaP cells as targets. Appropriate antigen processing and presentation of FPV-vectored rat PAP is assayed using PAP5-specific CTL. MoDC from HLA-A2.1⁺ donors are infected with rFPV.rPAP and antigen-specific reactivity determined by IFNγ-ELISPOT assay.

### EXAMPLE 5

### Xenoimmunization in a Rat Model

Each experimental group will comprise five eight week-old rat male Copenhagen rats. Rats are immunized with 2×10⁷ pfu of recombinant viral vectors intravenously (IV) or intramuscularly (IM). Four weeks later, rats are killed and tissues harvested. Sera are analysed by direct ELISA for the presence of anti-PAP antibodies. Prostate glands are examined histologically for evidence of autoimmune prostatitis. Single cell suspensions are prepared from spleens for in vitro recall proliferation and cytotoxicity assays as described in the study by Fong et al. We have obtained from Dr Fong the AT-1 and AT-3 cells, which are syngeneic to Copenhagen rats and are PAP-negative and PAP-positive, respectively.

As a positive control for the induction of autoimmune prostatitis, rats are immunized with the recombinant vaccinia vectors (rVV) that express human PAP (rVV.hPAP). Wild type virus and the recombinant virus that encodes rat PAP are used as negative controls. Virus vectors are available from: (i) Dendreon Corp. (Seattle, Wash., USA) and published by Fong et al.; (ii) Dr Doug McNeel, which remain unpublished.

The recombinant fowlpox virus vector that encodes (rFPV.hPAP) is tested for its ability to induce anti-PAP immune responses and autoimmune prostatitis.

The recombinant fowlpox virus vector that co-expresses human PAP and human IL-2 (rFPV.rPAP/hIL-2) is also tested.

Rats may also be primed with plasmid DNA that encodes human PAP (pcDNA3.1-hPAP) 100 μg IM three weeks before boosting with fowlpox virus vectors. Analysis is performed approximately four weeks after boosting.

### EXAMPLE 6

### VIR501 Comprising Rat PAP and IL-2 and VIR502 Comprising Human PAP and IL-2

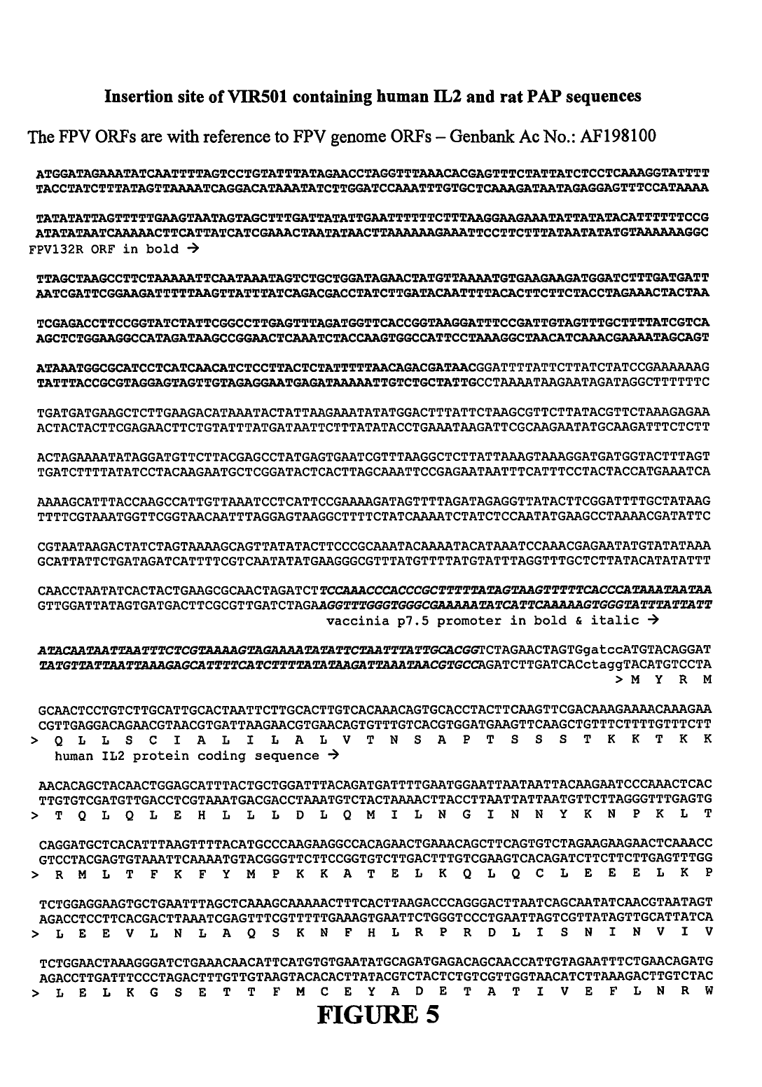

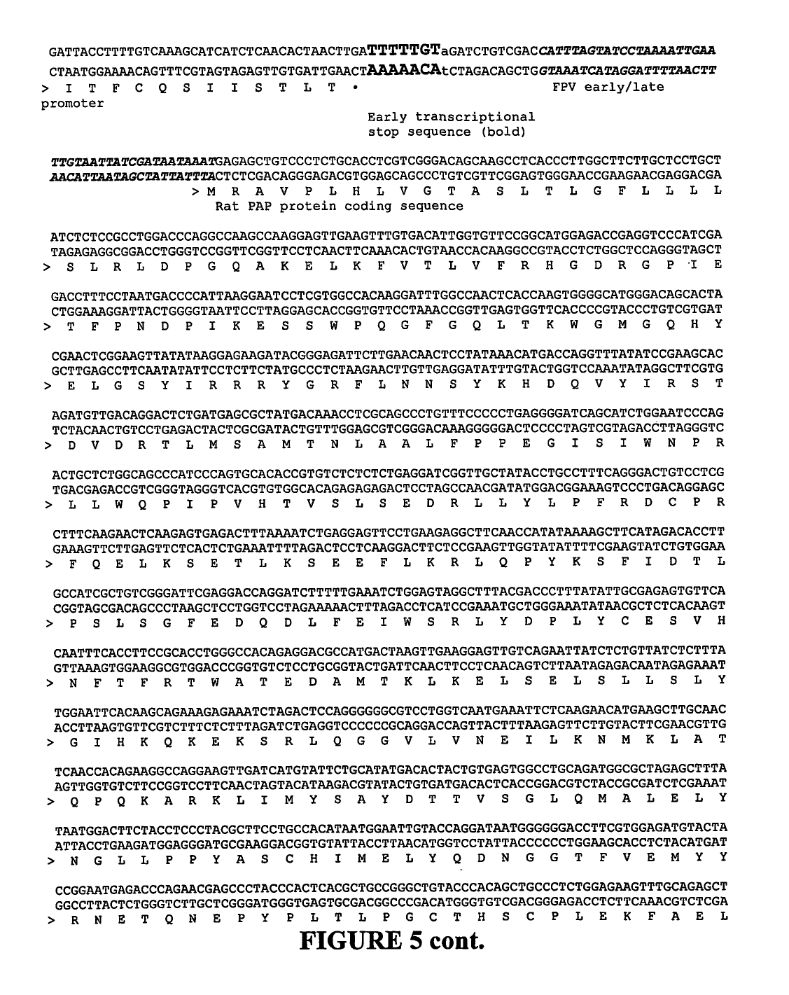

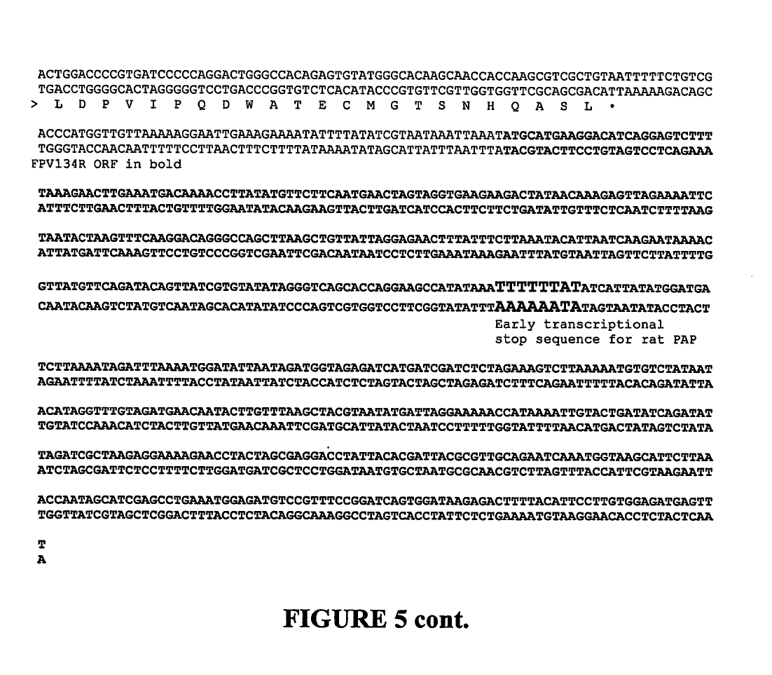

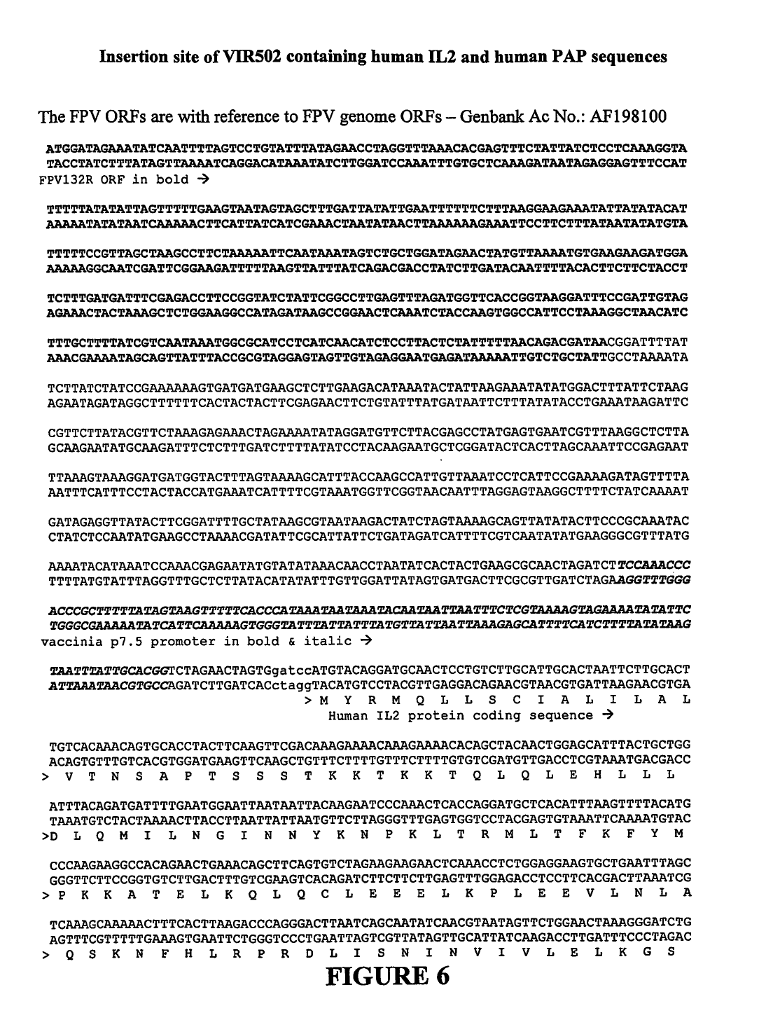

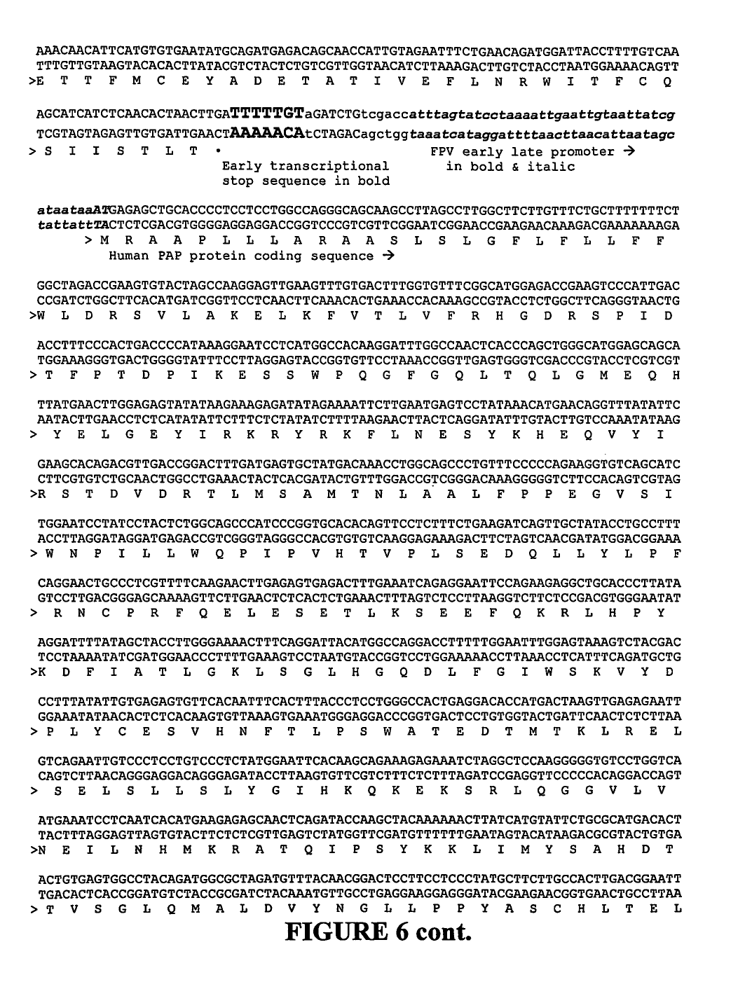

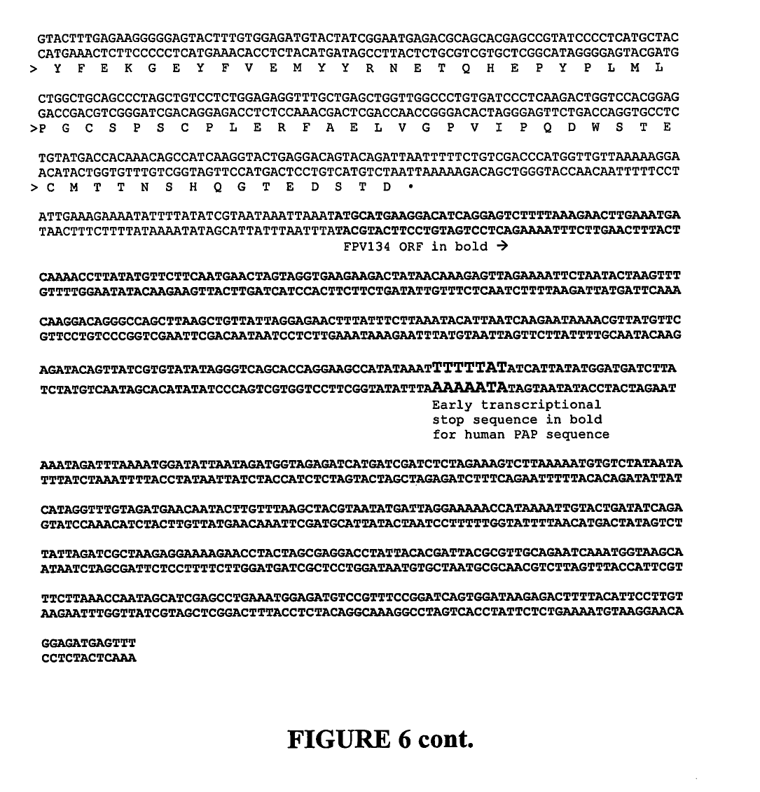

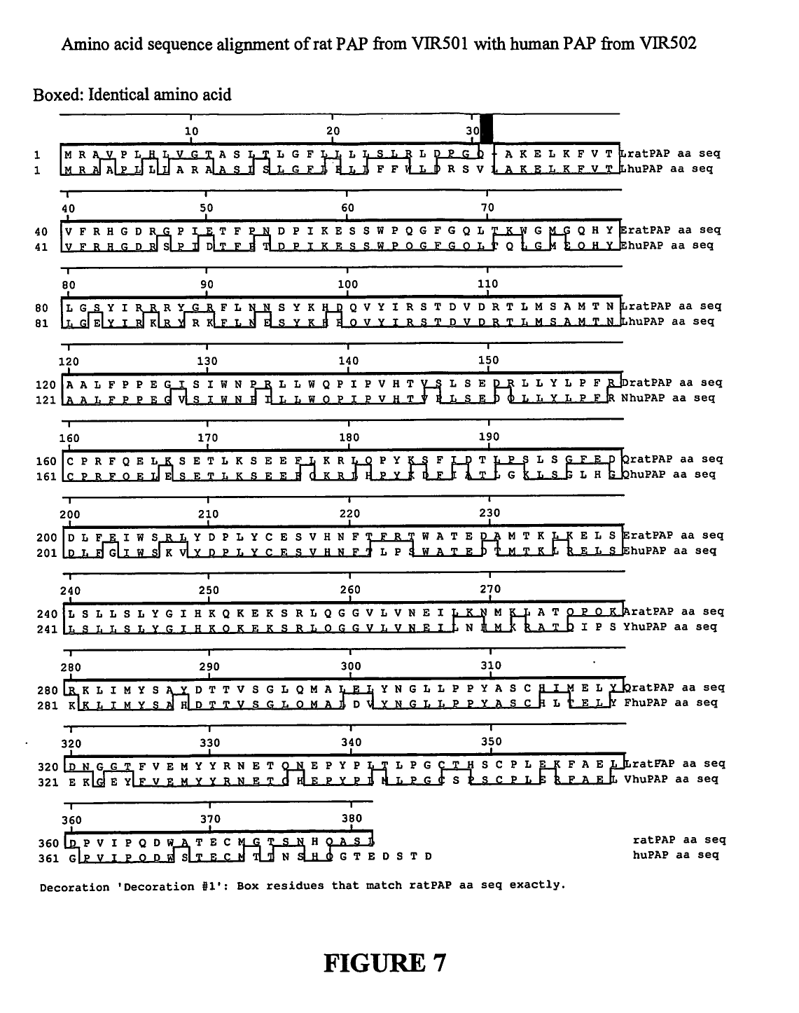

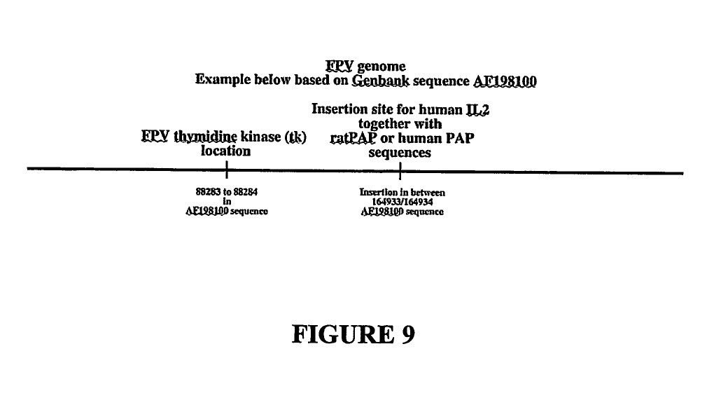

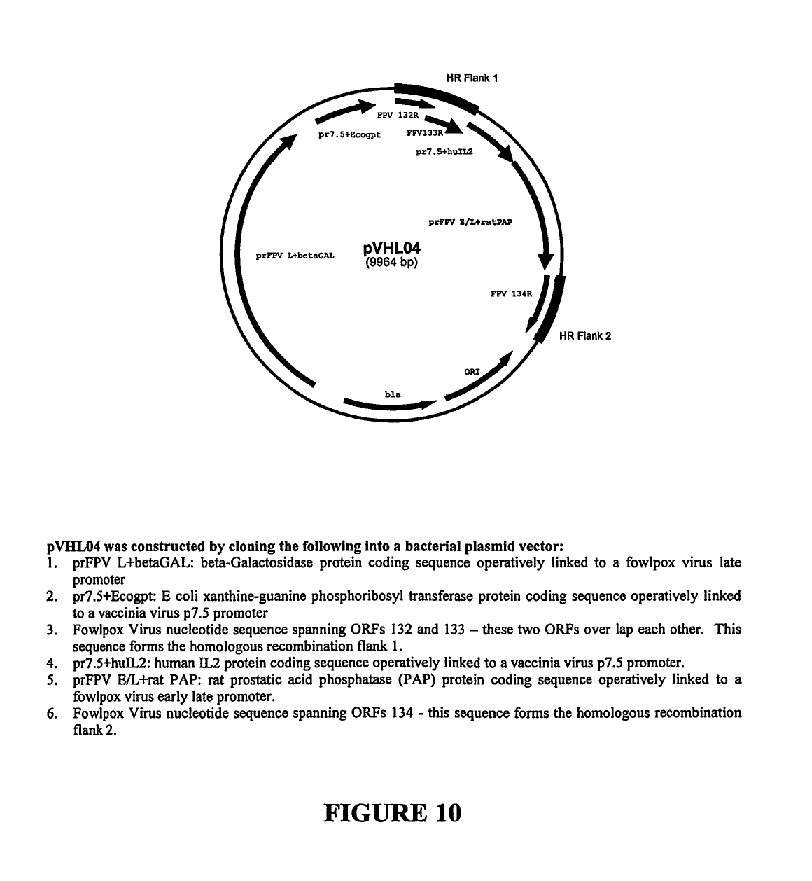

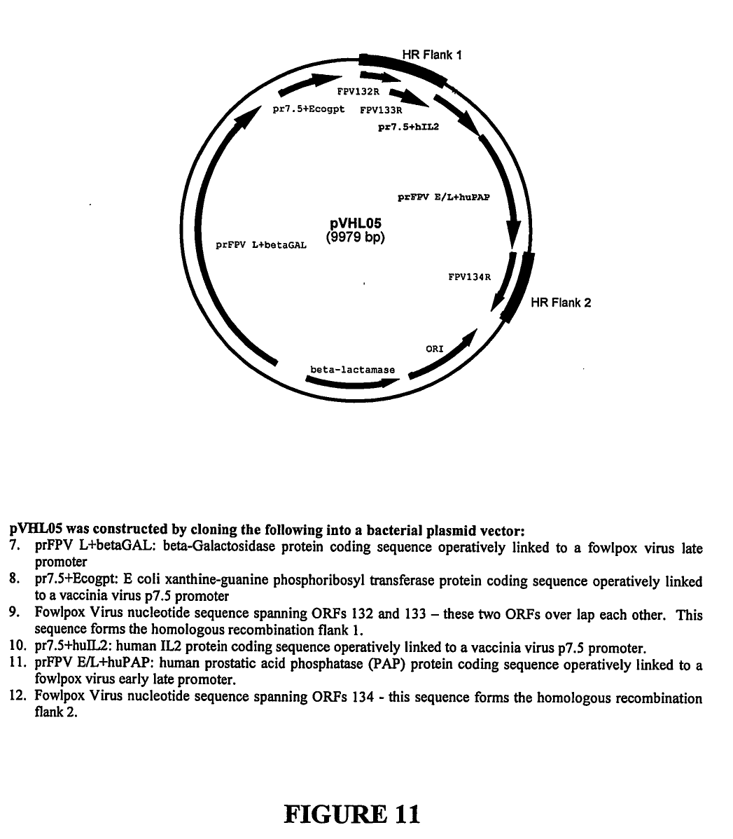

Further recombinant FPV vectors were constructed and tested as follows. Specifically, VIR501 comprising rat PAP and human IL-2 and VIR502 comprising human PAP and human IL-2 were generated in recombinant Fowlpox M3 essentially as described hereinbefore except that the integration vector comprised both inserts under the control of separate promoters. FIG. 9 shows the insertion in FPV relative to the FPV thymidine kinase gene. Integration vectors comprising cassettes for PAP and IL-2 were constructed with expression under the control of vaccinia virus p7.5 (human IL-2) and fowlpox virus early late promoter (rat PAP and human PAP). Plasmid maps of the integration vectors are set out in FIGS. 10 and 11. The nucleotide sequences of the insertion sites for VIR501 and VIR502 are set out in FIGS. 5 and 6 and in SEQ ID NO: 1, 2, 3 and 4. The amino acid sequences of rat and human PAP encoded by the vectors are set out in SEQ ID NO: 5 and 6 and are aligned in FIG. 7. The amino acid sequence of human IL-2 encoded by both vectors is set out in FIGS. 5 and 6 and in SEQ ID NO: 7.

### EXAMPLE 7

### VIR501 and VIR502 Express IL-2

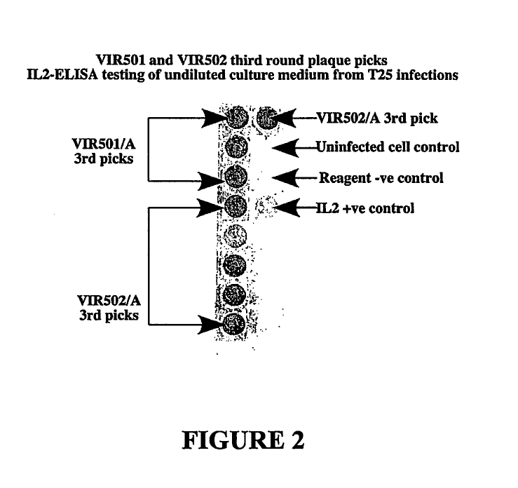

The vectors were tested, by ELISA, for their ability to express human IL-2. After three rounds of plaque purification, clones were amplified in CEF cells. After infection, culture media were tested for the presence of IL-2 using a human IL-2 ELISA kit. The results are shown in FIG. 2 where a visible colour change in the well indicates the presence of IL-2 in test wells.

### EXAMPLE 8

### TK143B Cells Infected with VIR501 or VIR502 Express PAP

One million cells of the thymidine kinase (TK)-deficient human osteosarcoma cell line, 143B, were plated in a 25 cm² flask overnight in complete DMEM with 10% FCS. Cells were infected with FPV vectors at a multiplicity of infection (MOI) of 10 for 48 h in a humidified 37° C. incubator in 5% CO₂ in air. The vectors used for infection were VIR501 (FPV encoding rat PAP and human IL-2), VIR502 (FPV encoding human PAP and human IL-2), FPV encoding influenza hamagglutinin (FPV-HA) as a negative control. At 48 h and 72 h post infection, the infected cells were harvested and lysed with lysis buffer (0.15M NaCl, 5 mM EDTA, 1% Triton X100, 10 mM Tris pH 8, 5 mM DTT and 100 PM PMSF). A clear lysate was collected after centrifugation at 12000 g for 15 min at 4° C. The samples were boiled in sample buffer and separated using 12% SDS-PAGE. The blot was transferred to transferred to a PVDF membrane (Amersham Pharmacia Biotech, Buckinghamshire, England), which was blocked with 2% bovine serum albumin (BSA) in PBS for 1 h at room temperature. After discarding the blocking buffer, the membrane was probed with polyclonal rabbit anti-human PAP (Signet Pathology System, Mass., USA) at 1:500 dilution in blocking buffer overnight at 4° C. After washing with 0.05% Tween-20 in PBS, a F(ab′)₂ fragment of goat anti-rabbit antibody conjugated to alkaline phosphatase (Jackson Immunoresearch, PA, USA) was applied at a dilution of 1:2000 and incubated for 1 h at room temperature. The protein bands were detected with the ECL substrate (Amersham Pharmacia Biotech, Buckinghamshire, England) and the fluorescent products were scanned using a Molecular Dynamics FluorImager.

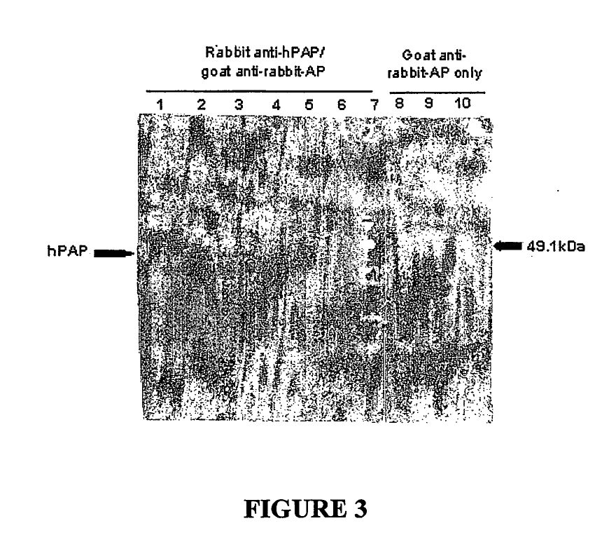

The Western blot (see FIG. 3) shows expression of human PAP from a VIR502 (FPV.hPAP)-infected human cell line. The polyclonal anti-human PAP antibody used for its detection does not cross-react with rat PAP (lane 6). However, the specific reaction of sheep immunised with VIR501 as described in Example 9 indicated that VIR501 successfully expresses rat PAP.

### EXAMPLE 9

### Immunogenicity of FPV Vectors Expressing Human PAP

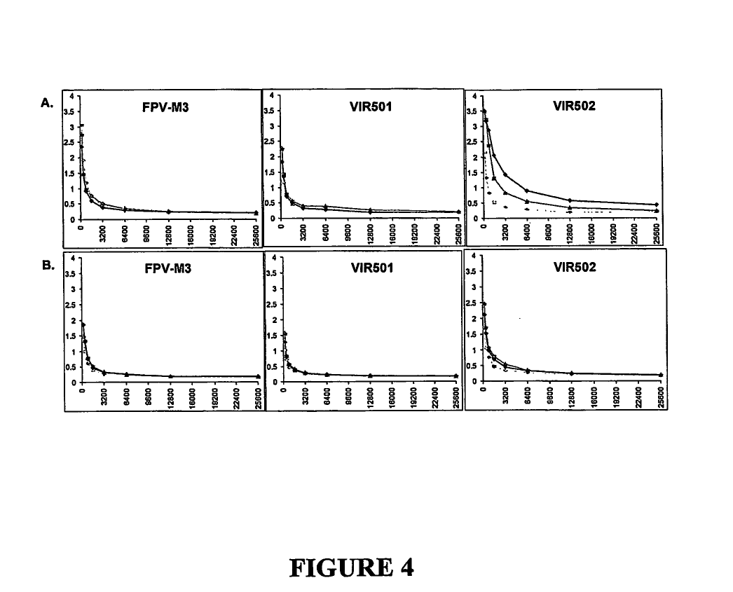

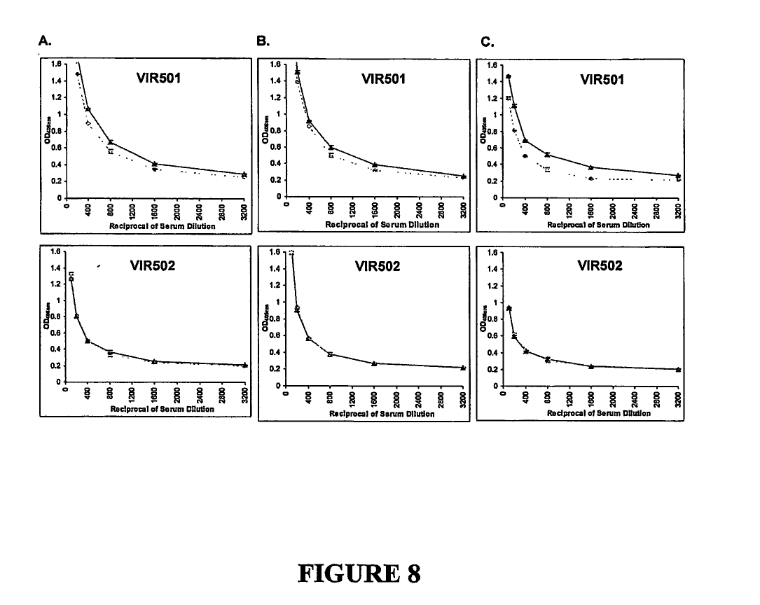

Bacterial recombinant proteins, pQE-hPAP or pQE-rPAP, or insect cell-derived human and rat PAP recombinant proteins (produced by stable transfection of the Sf21 Drosophila cell line using the InsectSelect™ expression system; Invitrogen, CA, USA) were diluted in 0.03M bicarbonate buffer (pH 9.6) at a concentration of 5 μg/mL and used to coat Maxisorp microtitre plates (Nunc, Roskilde, Denmark) overnight at 4° C. In each case, recombinant proteins were hexa-his tagged and purified on a Nickel affinity column. The plates were blocked with 2% BSA in PBS for 1 h at 37° C. (FIG. 4) or with 5% normal horse serum in PBS-azide for 1 h at 37° C. (FIG. 8) and then incubated with serial dilutions of rabbit sera in blocking buffer for 2 h at 37° C. (FIG. 4) or with serial dilutions of sheep sera in blocking buffer for 3 h at 37° C. (FIG. 8). After washing with 0.05% tween-20 in PBS, 1:2500 dilutions of alkaline phosphatase-conjugates of either goat anti-rabbit IgG [F(ab′)₂] (FIG. 4) or donkey anti-sheep IgG [F(ab′)₂] (FIG. 8) were added and incubated for a further 2 h. Bound antibodies were detected via hydrolysis of p-nitrophenyl phosphate substrate, and the developed colour was measured at OD 405 nm.

ELISA data (see FIG. 4) show a humoral immune response of male rabbits primarily to human PAP only after immunisation with VIR502. Presumably, because they are rodents, male rabbits are tolerant to the more closely related rat PAP encoded by VIR501. FIG. 8 shows the breakdown of tolerance when castrated male sheep were immunised with VIR501 or VIR502. Sheep immunised with VIR501 (rat PAP, human IL-2) responded to human PAP and rat PAP and the response was detectably enhanced when sera were tested against recombinant protein generated in insect cells. Over the time course assessed, sheep immunised with VIR502 (human PAP, human IL-2) did not generate as great a response to human PAP or rat PAP as that shown by sheep immunised with VIR501 (rat PAP, human IL-2). These data complement the data in rabbits, indicating that male sheep may be tolerant to human PAP rather than rat PAP. As PAP is prostate specific, a prostate specific immune response has been demonstrated after xenogeneic immunisation with VIR501 or VIR502.

ELISpot data confirm a human cellular immune response in vitro to VIR501. Human HLA-A2.1⁺ human monocyte-derived dendritic cells (MoDC) were generated by standard means in GM-CSF and IL-4. MoDC were matured using lipopolysaccharide for 24 hours and then infected for 5 days with VIR501 at a multiplicity of infection (MOI) of 10. Peripheral blood mononuclear cells (PBMC) from the same donor were co-cultured for 7 days with the VIR501-infected MoDC and then Ficoll-purified before an 18-hour interferon-γ (IFNγ) ELISpot assay using as antigen presenting cells (APC) either peptide-pulsed HLA-A2.1-expressing murine T2 cells or vaccinia virus-infected HLA-A2.1⁺ PBMC. In comparison with control APC (autologous PBMC without APC; T2 cells without peptide; T2 cells pulsed with an irrelevant HLA-A2 binding peptide from HTLV-1), T2 cells pulsed with HLA-A2.1-restricted PAP5 peptide (Peshwa et al. Prostate 129-138, 1998), which is identical between rat and human PAP, elicited a four-fold increase in the number of IFNγ⁺ spot forming cells (SFC). Moreover, in comparison with control APC (PBMC infected with wild type vaccinia), PBMC infected with vaccinia expressing rat or human PAP elicited an approximate three-fold increase in the number of IFNγ⁺ SFC. These data support the notion that after VIR501 infection, human APC correctly process and present endogenously expressed PAP protein to autologous T cells.

### BIBLIOGRAPHY

Albert, M. L. et al., Nature Medicine 4:1321-1324, 1998.
Altschul et al., Nucleic Acids Res. 25(17): 3389-3402, 1997.
Ausubel et al., “Current Protocols in Molecular Biology”, 5^{th} Edition, John Wiley & Sons, Inc., 2002.
Bonner et al., Eur. J. Biochem. 46(1): 83-88, 1974.
Boyle, D. B., et al., Gene 65(1):123-8, 1988.
Coupar, B. E. et al., Gene 68(1): 1-10, 1988.
Cox, W. et al., Virology 195:845-850, 1993.
Estcourt, M. J. et al., International Immunology 14(1):31-7, 2002.
Fong, L. et al., Journal of Immunology 159:3113-3117, 1997.
Fong, L. et al., J Immunol 167:7150-7156, 2001.
Kita, M. et al., Endocrinology 140:1392-1398, 1991.
Lee, P. P. et al., Nature Medicine 5(6):-677-685, 1999.
Lodge, P. A. et al., Cancer Research 60:829-833, 2000.
Logothetis, C. J. et al., Semin. Oncol. 21:620, 1994.
McNeel D. G. et al., J. Urinol. 164(5):1825-1839, 2000.
McNeel D. G. et al., Cancer Research 61:5161-5167, 2001.
Marmur et al., J. Mol. Biol. 5:109, 1962.
Murphy et al., Prostate 38:73-78, 1999(a).
Murphy, G. P. et al., Prostate 39:54-59, 1999(b).
Parker, K. C. et al., Journal of Immunology 152:163-175, 1994.
Perkus, M. E. et al., Virology 179:276-28, 1990.
Peshwa, M. V. et al., Prostate 36:129-138, 1998.
Rosenberg, S. A. et al., J Am Med Assoc 271:903, 1994.
Rosenberg, S. A. et al., Immunity 10:281-287, 1999.
Sambrook et al. “Molecular Cloning: A Laboratory Manual” Cold Spring Harbor Laboratory, 3rd Edition, 2001.
Santen R. J., J Clin. Endocrinal. Metab. 75:685-689, 1992.
Smith G. L., Chapter 9, Expression of genes by vaccinia virus. In Molecular Virology, A Practical Approach. Ed. A J Davison and R M Elliott. Practical Approach Series, IRL Press at Oxford University Press., 257-283, 1993.
Somogyi P. et al., Virology 197:439-444, 1993.
Taylor, J. et al., Vaccine 6:497-503, 1988.
Thenot, S. et al., Mol. Cell Endocrinol. 156:85-93, 1999.

Those skilled in the art will appreciate that the invention disclosed herein is susceptible to variations and modifications other than those specifically described. It is to be understood that the invention includes all such variations and modifications. The invention also includes all steps, features, compositions referred to or indicated in this specification, individually or collectively, and any and all combinations of any two or more steps or features.

1. A genetic vaccine construct comprising an avipox virus vector which incorporates and, on administration to a subject, expresses in a cell of said subject, a sequence of nucleotides encoding a xenogeneic prostate specific polypeptide or a derivative or analogue thereof, wherein said virus vector does not productively infect said subject.

2. A genetic vaccine construct comprising an avipox virus vector which incorporates and, on administration to a subject, expresses in a cell of said subject, a sequence of nucleotides encoding a xenogeneic prostate specific polypeptide or a derivative or analogue thereof, and a sequence of nucleotides encoding an immunostimulatory polypeptide, wherein said avipox virus vector does not productively infect said subject.

3. The genetic vaccine construct of claim 1 or 2, wherein the prostate specific polypeptide is prostatic acid phosphatase or a derivative or analogue thereof.

4. The genetic vaccine construct of claim 3, wherein the subject is a human subject.

5. The genetic vaccine construct of claim 4, wherein the xenogeneic prostate specific polypeptide is rodent prostatic acid phosphatase.

6. The genetic vaccine construct of claim 5, wherein the rodent prostatic acid phosphatase is rat prostatic acid phosphatase.

7. The genetic vaccine construct of claim 2, wherein the immunostimulatory polypeptide is a cytokine.

8. The genetic vaccine construct of claim 7, wherein the cytokine is one or more of IL-2, IL-12, TNFα, IFNγ, IL-6, IL-4, IL-7 or GM-CSF.

9. The genetic vaccine construct of claim 8, wherein the cytokine is one or more of IL-2, IFNγ or IL-12.

10. The genetic vaccine construct of claim 9, wherein the cytokine is IL-2.

11. The genetic vaccine construct of claims 1 or 2, wherein the avipox virus vector is a fowlpox virus vector.

12. A composition comprising the genetic vaccine construct according to claims 1 or 2.

13. A composition consisting essentially of the genetic vaccine construct according to claims 1 or 2.

14. The composition of claim 12, wherein expression products of said genetic vaccine construct stimulate a prostate cell specific immune response.

15. The composition of claim 14, wherein prostate cell specific immune response is a PAP specific immune response.

16. The composition of claim 14, wherein the expression products of the genetic vaccine construct stimulate autoimmune prostatitis.

17. A recombinant vector for use in making the genetic vaccine construct according to claims 1 or 2 comprising:
i) avipox virus vector nucleic acid sequences comprising sites for homologous recombination with an avipox virus vector;
ii) one or more promoters; and
iii) a sequence of nucleotides encoding a xenogeneic prostate specific polypeptide.

18. A recombinant vector for use in making the genetic vaccine construct according to claim 2 comprising:
i) avipox virus vector nucleic acid sequences comprising sites for homologous recombination with an avipox virus vector;
ii) one or more promoters;
iii) a sequence of nucleotides encoding a xenogeneic prostate specific polypeptide; and
iv) a sequence of nucleotides encoding an immunostimulatory polypeptide.

19. A eukaryotic cell infected with a genetic vaccine construct according to claims 1 or 2.

20. An antibody capable of acting as a marker for the genetic vaccine construct which antibody recognizes epitopes uniquely formed in expression products of the genetic vaccine construct according to claims 1 or 2.

21. A nucleic acid probe comprising a complementary form of a contiguous sequence of nucleotides of all or part of the genetic vaccine construct according to claims 1 or 2 which specifically recognizes said genetic vaccine construct under appropriate hybridization conditions.

22. A method for stimulating or otherwise enhancing a prostate cell specific immune response in a subject comprising administration to the subject of an effective amount of a composition comprising a genetic vaccine construct comprising an avipox virus vector which incorporates and, on administration to a subject, expresses in a cell of said subject, a sequence of nucleotides encoding a xenogeneic prostate specific polypeptide or a derivative or analogue thereof, for a time and under conditions sufficient for expression products of said genetic vaccine construct to stimulate or otherwise enhance a prostate cell specific immune response, and wherein said avipox virus does not productively infect said subject.

23. A method for stimulating or otherwise enhancing a prostate cell specific immune response in a subject comprising administration to said subject of an effective amount of a composition comprising a genetic vaccine construct comprising an avipox virus vector which incorporates and, on administration to a subject, expresses in a cell of said subject, a sequence of nucleotides encoding a xenogeneic prostate specific polypeptide or a derivative or analogue thereof and a sequence of nucleotides encoding an immunostimulatory polypeptide, for a time and under conditions sufficient for expression products of said genetic vaccine construct to stimulate or otherwise enhance a prostate cell specific immune response, and wherein said avipox virus vector does not productively infect said subject and a sequence of nucleotides encoding an immunostimulatory polypeptide.

24. A method for immunotherapy and/or immunoprophylaxis of prostate cancer comprising administration of an effective amount of a composition comprising a genetic vaccine construct comprising an avipox virus vector which incorporates and, on administration to a subject, expresses in a cell of said subject, a sequence of nucleotides encoding a xenogeneic prostate specific polypeptide or a derivative or analogue thereof, wherein said vector does not productively infect said subject, and wherein expression products of said vector stimulate a prostate cell specific immune response effective in the treatment and/or prophylaxis of prostate cancer.

25. A method for immunotherapy and/or immunoprophylaxis of prostate cancer comprising administration of an effective amount of a composition comprising a genetic vaccine construct comprising an avipox virus vector which incorporates and, on administration to a subject, expresses in a cell of said subject, a sequence of nucleotides encoding a xenogeneic prostate specific polypeptide or a derivative or analogue thereof, and a sequence of nucleotides encoding an immunostimulatory polypeptide, wherein said vector does not productively infect said subject, and wherein expression products of said vector stimulate a prostate cell specific immune response effective in the treatment and/or prophylaxis of prostate cancer.

26. The method of any one of claims 22 to 25, wherein the prostate specific polypeptide is a prostatic acid phosphatase or a derivative or analogue thereof and the prostate cell specific immune response is a PAP specific response.

27. A method of claim 26, wherein the subject is a human.

28. The method of claim 27, wherein the prostate specific polypeptide is rodent prostatic acid phosphatase.

29. The method of claim 28, wherein the rodent prostatic acid phosphatase is rat prostatic acid phosphatase.

30. The method of claim 23 or 25, wherein the immunostimulatory polypeptide is a cytokine.

31. The method of claim 29, wherein the cytokine is one or more of cytokines IL-2, IL-12, TNF-α, IFNγ, IL-6, IL-4, IL-7 or GM-CSF.

32. The method of claim 31, wherein the cytokine is one or more of cytokines IL-2, IFNγ and/or IL-12.

33. The method of claim 32, wherein the cytokine is IL-2.

34. The method of any one of claims 22 to 25, wherein the avipox virus vector is a fowlpox virus vector.

35-47. (canceled)


SEQUENCE LISTING (11 sequences)

SEQ ID NO: 1 - DNA, 3271 bp, virus
atggatagaaatatcaattttagtcctgtatttatagaacctaggtttaaacacgagtttctattatctcctcaaaggtatttttatatattagtttttgaagtaatagtagctttgattatattgaattttttctttaaggaagaaatattatatacattttttccgttagctaagccttctaaaaattcaataaatagtctgctggatagaactatgttaaaatgtgaagaagatggatctttgatgatttcgagaccttccggtatctattcggccttgagtttagatggttcaccggtaaggatttccgattgtagtttgcttttatcgtcaataaatggcgcatcctcatcaacatctccttactctatttttaacagacgataacggattttattcttatctatccgaaaaaagtgatgatgaagctcttgaagacataaatactattaagaaatatatggactttattctaagcgttcttatacgttctaaagagaaactagaaaatataggatgttcttacgagcctatgagtgaatcgtttaaggctcttattaaagtaaaggatgatggtactttagtaaaagcatttaccaagccattgttaaatcctcattccgaaaagatagttttagatagaggttatacttcggattttgctataagcgtaataagactatctagtaaaagcagttatatacttcccgcaaatacaaaatacataaatccaaacgagaatatgtatataaacaacctaatatcactactgaagcgcaactagatcttccaaacccacccgctttttatagtaagtttttcacccataaataataaatacaataattaatttctcgtaaaagtagaaaatatattctaatttattgcacggtctagaactagtggatccatgtacaggatgcaactcctgtcttgcattgcactaattcttgcacttgtcacaaacagtgcacctacttcaagttcgacaaagaaaacaaagaaaacacagctacaactggagcatttactgctggatttacagatgattttgaatggaattaataattacaagaatcccaaactcaccaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgatttttgtagatctgtcgaccatttagtatcctaaaattgaattgtaattatcgataataaatgagagctgtccctctgcacctcgtcgggacagcaagcctcacccttggcttcttgctcctgctatctctccgcctggacccaggccaagccaaggagttgaagtttgtgacattggtgttccggcatggagaccgaggtcccatcgagacctttcctaatgaccccattaaggaatcctcgtggccacaaggatttggccaactcaccaagtggggcatgggacagcactacgaactcggaagttatataaggagaagatacgggagattcttgaacaactcctataaacatgaccaggtttatatccgaagcacagatgttgacaggactctgatgagcgctatgacaaacctcgcagccctgtttccccctgaggggatcagcatctggaatcccagactgctctggcagcccatcccagtgcacaccgtgtctctctctgaggatcggttgctatacctgcctttcagggactgtcctcgctttcaagaactcaagagtgagactttaaaatctgaggagttcctgaagaggcttcaaccatataaaagcttcatagacaccttgccatcgctgtcgggattcgaggaccaggatctttttgaaatctggagtaggctttacgaccctttatattgcgagagtgttcacaatttcaccttccgcacctgggccacagaggacgccatgactaagttgaaggagttgtcagaattatctctgttatctctttatggaattcacaagcagaaagagaaatctagactccaggggggcgtcctggtcaatgaaattctcaagaacatgaagcttgcaactcaaccacagaaggccaggaagttgatcatgtattctgcatatgacactactgtgagtggcctgcagatggcgctagagctttataatggacttctacctccctacgcttcctgccacataatggaattgtaccaggataatggggggaccttcgtggagatgtactaccggaatgagacccagaacgagccctacccactcacgctgccgggctgtacccacagctgccctctggagaagtttgcagagctactggaccccgtgatcccccaggactgggccacagagtgtatgggcacaagcaaccaccaagcgtcgctgtaatttttctgtcgacccatggttgttaaaaaggaattgaaagaaaatattttatatcgtaataaattaaatatgcatgaaggacatcaggagtcttttaaagaacttgaaatgacaaaaccttatatgttcttcaatgaactagtaggtgaagaagactataacaaagagttagaaaattctaatactaagtttcaaggacagggccagcttaagctgttattaggagaactttatttcttaaatacattaatcaagaataaaacgttatgttcagatacagttatcgtgtatatagggtcagcaccaggaagccatataaattttttatatcattatatggatgatcttaaaatagatttaaaatggatattaatagatggtagagatcatgatcgatctctagaaagtcttaaaaatgtgtctataatacataggtttgtagatgaacaatacttgtttaagctacgtaatatgattaggaaaaaccataaaattgtactgatatcagatattagatcgctaagaggaaaagaacctactagcgaggacctattacacgattacgcgttgcagaatcaaatggtaagcattcttaaaccaatagcatcgagcctgaaatggagatgtccgtttccggatcagtggataagagacttttacattccttgtggagatgagttt

SEQ ID NO: 2 - DNA, 3271 bp, virus
tacctatctttatagttaaaatcaggacataaatatcttggatccaaatttgtgctcaaagataatagaggagtttccataaaaatatataatcaaaaacttcattatcatcgaaactaatataacttaaaaaagaaattccttctttataatatatgtaaaaaaggcaatcgattcggaagatttttaagttatttatcagacgacctatcttgatacaattttacacttcttctacctagaaactactaaagctctggaaggccatagataagccggaactcaaatctaccaagtggccattcctaaaggctaacatcaaacgaaaatagcagttatttaccgcgtaggagtagttgtagaggaatgagataaaaattgtctgctattgcctaaaataagaatagataggcttttttcactactacttcgagaacttctgtatttatgataattctttatatacctgaaataagattcgcaagaatatgcaagatttctctttgatcttttatatcctacaagaatgctcggatactcacttagcaaattccgagaataatttcatttcctactaccatgaaatcattttcgtaaatggttcggtaacaatttaggagtaaggcttttctatcaaaatctatctccaatatgaagcctaaaacgatattcgcattattctgatagatcattttcgtcaatatatgaagggcgtttatgttttatgtatttaggtttgctcttatacatatatttgttggattatagtgatgacttcgcgttgatctagaaggtttgggtgggcgaaaaatatcattcaaaaagtgggtatttattatttatgttattaattaaagagcattttcatcttttatataagattaaataacgtgccagatcttgatcacctaggtacatgtcctacgttgaggacagaacgtaacgtgattaagaacgtgaacagtgtttgtcacgtggatgaagttcaagctgtttcttttgtttcttttgtgtcgatgttgacctcgtaaatgacgacctaaatgtctactaaaacttaccttaattattaatgttcttagggtttgagtggtcctacgagtgtaaattcaaaatgtacgggttcttccggtgtcttgactttgtcgaagtcacagatcttcttcttgagtttggagacctccttcacgacttaaatcgagtttcgtttttgaaagtgaattctgggtccctgaattagtcgttatagttgcattatcaagaccttgatttccctagactttgttgtaagtacacacttatacgtctactctgtcgttggtaacatcttaaagacttgtctacctaatggaaaacagtttcgtagtagagttgtgattgaactaaaaacatctagacagctggtaaatcataggattttaacttaacattaatagctattatttactctcgacagggagacgtggagcagccctgtcgttcggagtgggaaccgaagaacgaggacgatagagaggcggacctgggtccggttcggttcctcaacttcaaacactgtaaccacaaggccgtacctctggctccagggtagctctggaaaggattactggggtaattccttaggagcaccggtgttcctaaaccggttgagtggttcaccccgtaccctgtcgtgatgcttgagccttcaatatattcctcttctatgccctctaagaacttgttgaggatatttgtactggtccaaatataggcttcgtgtctacaactgtcctgagactactcgcgatactgtttggagcgtcgggacaaagggggactcccctagtcgtagaccttagggtctgacgagaccgtcgggtagggtcacgtgtggcacagagagagactcctagccaacgatatggacggaaagtccctgacaggagcgaaagttcttgagttctcactctgaaattttagactcctcaaggacttctccgaagttggtatattttcgaagtatctgtggaacggtagcgacagccctaagctcctggtcctagaaaaactttagacctcatccgaaatgctgggaaatataacgctctcacaagtgttaaagtggaaggcgtggacccggtgtctcctgcggtactgattcaacttcctcaacagtcttaatagagacaatagagaaataccttaagtgttcgtctttctctttagatctgaggtccccccgcaggaccagttactttaagagttcttgtacttcgaacgttgagttggtgtcttccggtccttcaactagtacataagacgtatactgtgatgacactcaccggacgtctaccgcgatctcgaaatattacctgaagatggagggatgcgaaggacggtgtattaccttaacatggtcctattacccccctggaagcacctctacatgatggccttactctgggtcttgctcgggatgggtgagtgcgacggcccgacatgggtgtcgacgggagacctcttcaaacgtctcgatgacctggggcactagggggtcctgacccggtgtctcacatacccgtgttcgttggtggttcgcagcgacattaaaaagacagctgggtaccaacaatttttccttaactttcttttataaaatatagcattatttaatttatacgtacttcctgtagtcctcagaaaatttcttgaactttactgttttggaatatacaagaagttacttgatcatccacttcttctgatattgtttctcaatcttttaagattatgattcaaagttcctgtcccggtcgaattcgacaataatcctcttgaaataaagaatttatgtaattagttcttattttgcaatacaagtctatgtcaatagcacatatatcccagtcgtggtccttcggtatatttaaaaaatatagtaatatacctactagaattttatctaaattttacctataattatctaccatctctagtactagctagagatctttcagaatttttacacagatattatgtatccaaacatctacttgttatgaacaaattcgatgcattatactaatcctttttggtattttaacatgactatagtctataatctagcgattctccttttcttggatgatcgctcctggataatgtgctaatgcgcaacgtcttagtttaccattcgtaagaatttggttatcgtagctcggactttacctctacaggcaaaggcctagtcacctattctctgaaaatgtaaggaacacctctactcaaa

SEQ ID NO: 3 - DNA, 3286 bp, virus
atggatagaaatatcaattttagtcctgtatttatagaacctaggtttaaacacgagtttctattatctcctcaaaggtatttttatatattagtttttgaagtaatagtagctttgattatattgaattttttctttaaggaagaaatattatatacattttttccgttagctaagccttctaaaaattcaataaatagtctgctggatagaactatgttaaaatgtgaagaagatggatctttgatgatttcgagaccttccggtatctattcggccttgagtttagatggttcaccggtaaggatttccgattgtagtttgcttttatcgtcaataaatggcgcatcctcatcaacatctccttactctatttttaacagacgataacggattttattcttatctatccgaaaaaagtgatgatgaagctcttgaagacataaatactattaagaaatatatggactttattctaagcgttcttatacgttctaaagagaaactagaaaatataggatgttcttacgagcctatgagtgaatcgtttaaggctcttattaaagtaaaggatgatggtactttagtaaaagcatttaccaagccattgttaaatcctcattccgaaaagatagttttagatagaggttatacttcggattttgctataagcgtaataagactatctagtaaaagcagttatatacttcccgcaaatacaaaatacataaatccaaacgagaatatgtatataaacaacctaatatcactactgaagcgcaactagatcttccaaacccacccgctttttatagtaagtttttcacccataaataataaatacaataattaatttctcgtaaaagtagaaaatatattctaatttattgcacggtctagaactagtggatccatgtacaggatgcaactcctgtcttgcattgcactaattcttgcacttgtcacaaacagtgcacctacttcaagttcgacaaagaaaacaaagaaaacacagctacaactggagcatttactgctggatttacagatgattttgaatggaattaataattacaagaatcccaaactcaccaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgatttttgtagatctgtcgaccatttagtatcctaaaattgaattgtaattatcgataataaatgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaacttatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccctagctgtcctctggagaggtttgctgagctggttggccctgtgatccctcaagactggtccacggagtgtatgaccacaaacagccatcaaggtactgaggacagtacagattaatttttctgtcgacccatggttgttaaaaaggaattgaaagaaaatattttatatcgtaataaattaaatatgcatgaaggacatcaggagtcttttaaagaacttgaaatgacaaaaccttatatgttcttcaatgaactagtaggtgaagaagactataacaaagagttagaaaattctaatactaagtttcaaggacagggccagcttaagctgttattaggagaactttatttcttaaatacattaatcaagaataaaacgttatgttcagatacagttatcgtgtatatagggtcagcaccaggaagccatataaattttttatatcattatatggatgatcttaaaatagatttaaaatggatattaatagatggtagagatcatgatcgatctctagaaagtcttaaaaatgtgtctataatacataggtttgtagatgaacaatacttgtttaagctacgtaatatgattaggaaaaaccataaaattgtactgatatcagatattagatcgctaagaggaaaagaacctactagcgaggacctattacacgattacgcgttgcagaatcaaatggtaagcattcttaaaccaatagcatcgagcctgaaatggagatgtccgtttccggatcagtggataagagacttttacattccttgtggagatgagttt

SEQ ID NO: 4 - DNA, 3286 bp, virus
tacctatctttatagttaaaatcaggacataaatatcttggatccaaatttgtgctcaaagataatagaggagtttccataaaaatatataatcaaaaacttcattatcatcgaaactaatataacttaaaaaagaaattccttctttataatatatgtaaaaaaggcaatcgattcggaagatttttaagttatttatcagacgacctatcttgatacaattttacacttcttctacctagaaactactaaagctctggaaggccatagataagccggaactcaaatctaccaagtggccattcctaaaggctaacatcaaacgaaaatagcagttatttaccgcgtaggagtagttgtagaggaatgagataaaaattgtctgctattgcctaaaataagaatagataggcttttttcactactacttcgagaacttctgtatttatgataattctttatatacctgaaataagattcgcaagaatatgcaagatttctctttgatcttttatatcctacaagaatgctcggatactcacttagcaaattccgagaataatttcatttcctactaccatgaaatcattttcgtaaatggttcggtaacaatttaggagtaaggcttttctatcaaaatctatctccaatatgaagcctaaaacgatattcgcattattctgatagatcattttcgtcaatatatgaagggcgtttatgttttatgtatttaggtttgctcttatacatatatttgttggattatagtgatgacttcgcgttgatctagaaggtttgggtgggcgaaaaatatcattcaaaaagtgggtatttattatttatgttattaattaaagagcattttcatcttttatataagattaaataacgtgccagatcttgatcacctaggtacatgtcctacgttgaggacagaacgtaacgtgattaagaacgtgaacagtgtttgtcacgtggatgaagttcaagctgtttcttttgtttcttttgtgtcgatgttgacctcgtaaatgacgacctaaatgtctactaaaacttaccttaattattaatgttcttagggtttgagtggtcctacgagtgtaaattcaaaatgtacgggttcttccggtgtcttgactttgtcgaagtcacagatcttcttcttgagtttggagacctccttcacgacttaaatcgagtttcgtttttgaaagtgaattctgggtccctgaattagtcgttatagttgcattatcaagaccttgatttccctagactttgttgtaagtacacacttatacgtctactctgtcgttggtaacatcttaaagacttgtctacctaatggaaaacagtttcgtagtagagttgtgattgaactaaaaacatctagacagctggtaaatcataggattttaacttaacattaatagctattatttactctcgacgtggggaggaggaccggtcccgtcgttcggaatcggaaccgaagaacaaagacgaaaaaaagaccgatctggcttcacatgatcggttcctcaacttcaaacactgaaaccacaaagccgtacctctggcttcagggtaactgtggaaagggtgactggggtatttccttaggagtaccggtgttcctaaaccggttgagtgggtcgacccgtacctcgtcgtaatacttgaacctctcatatattctttctctatatcttttaagaacttactcaggatatttgtacttgtccaaatataagcttcgtgtctgcaactggcctgaaactactcacgatactgtttggaccgtcgggacaaagggggtcttccacagtcgtagaccttaggataggatgagaccgtcgggtagggccacgtgtgtcaaggagaaagacttctagtcaacgatatggacggaaagtccttgacgggagcaaaagttcttgaactctcactctgaaactttagtctccttaaggtcttctccgacgtgggaatattcctaaaatatcgatggaacccttttgaaagtcctaatgtaccggtcctggaaaaaccttaaacctcatttcagatgctgggaaatataacactctcacaagtgttaaagtgaaatgggaggacccggtgactcctgtggtactgattcaactctcttaacagtcttaacagggaggacagggagataccttaagtgttcgtctttctctttagatccgaggttcccccacaggaccagttactttaggagttagtgtacttctctcgttgagtctatggttcgatgttttttgaatagtacataagacgcgtactgtgatgacactcaccggatgtctaccgcgatctacaaatgttgcctgaggaaggagggatacgaagaacggtgaactgccttaacatgaaactcttccccctcatgaaacacctctacatgatagccttactctgcgtcgtgctcggcataggggagtacgatggaccgacgtcgggatcgacaggagacctctccaaacgactcgaccaaccgggacactagggagttctgaccaggtgcctcacatactggtgtttgtcggtagttccatgactcctgtcatgtctaattaaaaagacagctgggtaccaacaatttttccttaactttcttttataaaatatagcattatttaatttatacgtacttcctgtagtcctcagaaaatttcttgaactttactgttttggaatatacaagaagttacttgatcatccacttcttctgatattgtttctcaatcttttaagattatgattcaaagttcctgtcccggtcgaattcgacaataatcctcttgaaataaagaatttatgtaattagttcttattttgcaatacaagtctatgtcaatagcacatatatcccagtcgtggtccttcggtatatttaaaaaatatagtaatatacctactagaattttatctaaattttacctataattatctaccatctctagtactagctagagatctttcagaatttttacacagatattatgtatccaaacatctacttgttatgaacaaattcgatgcattatactaatcctttttggtattttaacatgactatagtctataatctagcgattctccttttcttggatgatcgctcctggataatgtgctaatgcgcaacgtcttagtttaccattcgtaagaatttggttatcgtagctcggactttacctctacaggcaaaggcctagtcacctattctctgaaaatgtaaggaacacctctactcaaa

SEQ ID NO: 5 - protein, 381 aa, rat
MRAVPLHLVGTASLTLGFLLLLSLRLDPGQAKELKFVTLVFRHGDRGPIETFPNDPIKESSWPQGFGQLTKWGMGQHYELGSYIRRRYGRFLNNSYKHDQVYIRSTDVDRTLMSAMTNLAALFPPEGISIWNPRLLWQPIPVHTVSLSEDRLLYLPFRDCPRFQELKSETLKSEEFLKRLQPYKSFIDTLPSLSGFEDQDLFEIWSRLYDPLYCESVHNFTFRTWATEDAMTKLKELSELSLLSLYGIHKQKEKSRLQGGVLVNEILKNMKLATQPQKARKLIMYSAYDTTVSGLQMALELYNGLLPPYASCHIMELYQDNGGTFVEMYYRNETQNEPYPLTLPGCTHSCPLEKFAELLDPVIPQDWATECMGTSNHQASL

SEQ ID NO: 6 - protein, 386 aa, human
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 7 - protein, 156 aa, human
MYRMQLLSCIALILALVTNSAPTSSSTKKTKKTQLQLEHLLLDLQMILNGINNYKNPKLTRMLTFKFYMPKKATELKQLQCLEEELKPLEEVLNLAQSKNFHLRPRDLISNINVIVLELKGSETTFMCEYADETATIVEFLNRWITFCQSIISTLT

SEQ ID NO: 8 - DNA, 30 bp, rat
ctctgaggatcggttgctatacctgccttt

SEQ ID NO: 9 - DNA, 30 bp, rat
acttagtcatggcgtcctctgtggcccagg

SEQ ID NO: 10 - DNA, 30 bp, human
ttcaggattacatggccaggacctttttgg

SEQ ID NO: 11 - DNA, 30 bp, human
ctcagtaccttgatggctgtttgtggtcat